(19) United States
(12) Patent Application Publication
(10) Pub. No.: US 2022/0202950 A1
(43) Pub. Date: Jun. 30, 2022

(54) COMPOSITIONS COMPRISING BACTERIALLY DERIVED INTACT MINICELLS FOR THERANOSTIC APPLICATIONS

(71) Applicant: EnGeneIC Molecular Delivery Pty Ltd, Sydney (AU)

(72) Inventors: Himanshu Brahmbhatt, Killara, Sydney (AU); Jennifer MacDiarmid, Sydney (AU)

(73) Assignee: EnGeneIC Molecular Delivery Pty Ltd, Sydney (AU)

(21) Appl. No.: 17/607,000

(22) PCT Filed: Apr. 30, 2020
(86) PCT No.: PCT/IB2020/054086

§ 371 Date: Oct. 27, 2021

Related U.S. Application Data
(60) Provisional application No. 62/841,828, filed on May 1, 2019.

(51) Int. Cl. A61K 47/69; A61K 35/74; A61K 51/12; A61P 35/00

(52) U.S. Cl. CPC A61K 47/6901; A61P 35/00; A61K 51/1203; A61K 35/74

(57) ABSTRACT

This disclosure relates generally to compositions and methods for treating cancer. The compositions comprise bacterially derived intact minicells or intact killed bacterial cells.

### CROSS-REFERENCE TO RELATED APPLICATION

This application claims priority to U.S. Provisional Application No. 62/841,828, filed May 1, 2019, the disclosure of which is specifically incorporated by reference in its entirety.

### FIELD

The present application relates generally to compositions and methods for treating cancer. The compositions comprise bacterially derived intact minicells.

### BACKGROUND

Cancer remains one of the most devastating diseases despite continuous development and innovation in cancer therapy. Surgery, radiotherapy and chemotherapy are the key components of cancer treatment. Currently, most drugs used for treating cancer are administered systemically. Although systemic delivery of cytotoxic anticancer drugs plays a crucial role in cancer therapeutics, it also engenders serious problems. For instance, systemic exposure of normal tissues/organs to the administered drug can cause severe toxicity. This is exacerbated by the fact that systemically delivered cancer chemotherapy drugs often must be delivered at very high dosages to overcome poor bioavailability of the drugs and the large volume of distribution within a patient. Also, systemic drug administration can be invasive, as it often requires the use of a secured catheter in a major blood vessel. Because systemic drug administration often requires the use of veins, either peripheral or central, it can cause local complications such as phlebitis. Extravasation of a drug also can lead to vesicant/tissue damage at the local site of administration, such as is commonly seen upon administration of vinca alkaloids and anthracyclines.

Nanomedicine has applied nanotechnology in various medical fields such as imaging in diagnosis of or therapy in human diseases. Theranostics combines the last two fields, while theranostic nanomedicine produces “nanoparticle-based drugs” simultaneously capable of the diagnosis and treatment of a disease. Theranostics can also be developed where nanoparticles or peptides carry diagnostic radionuclides that emit a “curative” type of radiations such as beta- or alpha-particles [Mango and Pacilio, 2016].

Theranostics is a new field of medicine which combines specific targeted therapy based on specific targeted diagnostic tests. With a key focus on patient centered care, theranostics provides a transition from conventional medicine to a contemporary personalized and precision medicine approach. The theranostics paradigm involves using nanoscience to unite diagnostic and therapeutic applications to form a single agent, allowing for diagnosis, drug delivery and treatment response monitoring. Theranostics uses specific biological pathways in the human body, to acquire diagnostic images and also to deliver a therapeutic dose of radiation to the patient. A specific diagnostic test shows a particular molecular target on a tumor, allowing a therapeutic agent to specifically target that receptor on the tumor, rather than more broadly the disease and location it presents. This contemporary form of treatment moves away from the one-medicine-fits-all and trial and error medicine approach, to offering the right treatment, for the right patient, at the right time, with the right dose, providing a more targeted, efficient pharmacotherapy in the form of theranostics.

Nanoparticles could be modified with imaging components to produce theranostic systems that enable non-invasive, real-time monitoring of drug delivery and therapeutic response. Of the theranostic nanoparticles being studied, superparamagnetic iron oxide nanoparticles (SPIONS) have been appealing owing to their intrinsic super paramagnetism that provides contrast in magnetic resonance imaging (MRI), and solid core to which therapeutics can be easily arranged. Iron oxide has been known to be biocompatible and biodegradable and a number of drug loaded theranostic SPIONs have been investigated.

Despite the promise of these theranostic nanoparticles, fabrication of reproducible and consistent formulations with controlled drug loading and release profiles remains a significant challenge and a major barrier to their clinical application. The difficulty lies in fabrication schemes that involve complex, multi-step synthesis procedures that can multiply and accumulate the variations or fluctuations from each step, leading to significant batch-to-batch inconsistencies and inefficient drug loading.

While bacterial minicells have been previously described, prior disclosures do not entail a theranostic, where a combination of a therapeutic agent is used in conjunction with a diagnostic agent. See e.g., US 2018-0043027 A1.

Accordingly, there remains a great need for new theranostic delivery systems. The present invention satisfies this need.

### SUMMARY OF THE INVENTION

One embodiment of the invention relates to a theranostic composition comprising: (a) a plurality of purified, intact bacterially derived minicells or intact killed bacterial cells; (b) at least one anti-neoplastic agent comprised within the minicells or killed cells; (c) a bispecific ligand, wherein the bispecific ligand is attached to a first surface component of the minicells or killed cells, and wherein the bispecific ligand comprises a first arm with binding specificity for the first surface component, and a second arm with binding specificity for a tumor cell surface receptor; and (d) a monospecific ligand attached to a second surface component of the minicells, wherein the monospecific ligand has at least one radio-imaging agent conjugated to the monospecific ligand at one or more conjugation residues of the monospecific ligand.

In one embodiment, the bispecific ligand and/or the monospecific ligand comprise a polypeptide, aptamer, carbohydrate, or a combination thereof. In another embodiment, the bispecific ligand has a specificity to a non-phagocytic mammalian tumor cell surface receptor. The non-phagocytic mammalian tumor cell surface receptor can comprise a tumor cell antigen.

In one embodiment, the tumor cell surface receptor comprises an integrin, neuromedin B receptor, bombesin 3 receptor, GRP receptor, bombesin 4 receptor, CCK2/gastrin, melanocortin-1 receptor (MC-1r), neuropeptide Y (NPY) receptor, neutrotensin (NT) receptor, prostate specific membrane antigen (PSMA), somatostatin (SST) receptor, neurokinin 1 receptor (NK1R), chemokine receptor type 4 (CXCR4), vasoactive intestinal peptide (VIP), epidermal growth factor receptor (EGFR), vascular endothelial growth factor receptor (VEGFR), platelet-derived growth factor receptor (PDGFR), insulin-like growth factor receptor (IGFR), or any combination thereof. In another embodiment, the tumor cell surface receptor comprises EpCAM, CCR5, CD19, HER-2 neu, HER-3, HER-4, EGFR, PSMA, CEA, MUC-1 (mucin), MUC2, MUC3, MUC4, MUC5, MUC5, MUC7, BhcG, Lewis-Y, CD20, CD33, CD30, ganglioside GD3, 9-O-Acetyl-GD3, GM2, Globo H, fucosyl GM1, Poly SA, GD2, carboanhydrase IX (MN/CA IX), CD44v6, sonic hedgehog (Shh), Wue-1, Plasma Cell Antigen, (membrane-bound) IgE, melanoma chondroitin sulfate proteoglycan (MCSP), CCR8, TNF-alpha precursor, STEAP, mesothelin, A33 antigen, prostate stem cell antigen (PSCA), Ly-6; desmoglein 4, E-cadherin neoepitope, fetal acetylcholine receptor, CD25, CA19-9 marker, CA-125 marker and muellerian inhibitory substance (MIS) receptor type II, sTn (sialylated Tn antigen; TAG-72), FAP (fibroblast activation antigen), endosialin, EGFRVIII, LG, SAS, CD63, or any combination thereof.

In one embodiment, the bispecific ligand comprises an antibody that specifically recognizes the tumor cell antigen.

In another embodiment, the one or more conjugation residues are each independently selected from the group consisting of an F-amino group on a lysine side chain, a guanidinium group on an arginine side chain, a carboxyl group on an aspartic acid or glutamic acid, a cysteine thiol, a phenol on a tyrosine, and a combination thereof.

In one embodiment, the monospecific ligand has a variable length and comprises a polypeptide comprising from about 15 to about 500 amino acids. In another embodiment, the amount of radio-imaging agent conjugated to the monospecific ligand varies directly with the amount of conjugation residues. In yet another embodiment, the monospecific ligand can be increased in length to produce a corresponding increase in an amount of the radio-imaging agent. Further, the composition can comprise a diagnostically effective amount of radio-imaging agent sufficient to produce a clear image of the tumor upon radioimaging.

In another embodiment, the radio-imaging agent also functions as a therapeutic radiation emitting agent, wherein the amount of radiation emitted by the radio-imaging agent is sufficient to provide a therapeutic effect on the tumor. For example, the therapeutic effect can be a reduction in tumor size. The tumor can be reduced in size, for example, by about 100%, about 90%, about 80%, about 70%, about 60%, about 50%, about 40%, about 30%, about 20%, about 10%, or about 5%.

In one embodiment, the theranostic composition comprises about 30 Gy to about 100 Gy radiation. In another embodiment, the radio-imaging agent comprises a radioisotope, magnetic nanoparticle, organic fluorescent dye, or any combination thereof. For example, the radio-imaging agent can comprise a radioisotope selected from the group consisting of yttrium-90, yttrium-86, terbium-152, terbium-155, terbium-149, terbium-161, technetium-99m, iodine-123, iodine-131, rubidium-82, thallium-201, gallium-67, fluorine-18, copper-64, gallium-68, xenon-133, indium-111, lutetium-177, and any combination thereof. In another embodiment, the radio-imaging agent can be comprised within a synthetic nanoparticle, and wherein the synthetic nanoparticle is conjugated to the monospecific ligand.

In another embodiment, the radio-imaging agent can be conjugated to the monospecific ligand via a linker.

In a further embodiment, the minicell or killed bacterial cell comprises a pharmaceutically effective polymer film or coat. For example, the polymer film or coat can be opsonization-reducing. In another embodiment, the polymer film or coat reduces or minimizes macrophage uptake of the composition. Further, the polymer film or coat can comprise a polymer selected from the group consisting of a polyethylene glycol, polymer-PEO-blockpoly(γ-methacryloxypropyltrimethoxysilane) (PEOb-PγMPS), and (trimethoxysilyl)propyl methacrylate-PEG-methacrylate.

In another embodiment, the theranostic composition comprises a minicell or killed bacterial cell which is PEGylated.

In one embodiment, the bispecific ligand comprises Arg-Gly-Asp (RGD) peptide, bombesin (BBN)/gastrin-releasing peptide (GRP), cholecystokinin (CCK)/gastrin peptide, α-melanocyte-stimulating hormone (α-MSH), neuropeptide Y (NPY), neutrotensin (NT), [⁶⁸Ga]Ga-PSMA-HBED-CC ([⁶⁸Ga]Ga-PSMA-11 [PET]), [¹⁷⁷Lu]Lu/[⁹⁰Y]Y-J591, [123]I-MIP-1072, [¹³¹I]I-MIP-1095, ⁶⁸Ga or ¹⁷⁷Lu labeled PSMA-I&T, ⁶⁸Ga or ¹⁷⁷Lu labeled DKFZ-PSMA-617 (PSMA-617), somatostatin (SST) peptide, substance P, T140, tumor molecular targeted peptide 1 (TMTP1), vasoactive intestinal peptide (VIP), or any combination thereof.

In another embodiment, the second arm of the bispecific ligand comprises the Arg-Gly-Asp (RGD) peptide, bombesin (BBN)/gastrin-releasing peptide (GRP), cholecystokinin (CCK)/gastrin peptide, α-melanocyte-stimulating hormone (α-MSH), neuropeptide Y (NPY), neutrotensin (NT), [⁶⁸Ga]Ga-PSMA-HBED-CC ([⁶⁸Ga]Ga-PSMA-11 [PET]), [¹⁷⁷Lu]Lu/[90Y]Y-J591, [¹²³I]I-MIP-1072, [¹³¹I]I-MIP-1095, ⁶⁸Ga or ¹⁷⁷Lu labeled PSMA-I&T, ⁶⁸Ga or ¹⁷⁷Lu labeled DKFZ-PSMA-617 (PSMA-617), somatostatin (SST) peptide, substance P, T140, tumor molecular targeted peptide 1 (TMTP1), vasoactive intestinal peptide (VIP), or any combination thereof.

In one embodiment, the first minicell surface component and/or the second minicell surface component comprises a lipopolysaccharide (LPS). In another embodiment, an O-polysaccharide of the lipopolysaccharide can be radiolabeled.

In another embodiment, the theranostic composition comprises an anti-neoplastic agent which is a super-cytotoxic drug. For example, the super-cytotoxic drug can have an LD₅₀ that is lower than the ED₅₀ of the super-cytotoxic drug for a targeted cancer. An exemplary supertoxic antineoplastic agent is PNU-159682. In one embodiment, the minicell or killed cells comprise from about 5×10⁵ to about 1.5×10⁶ molecules of the super-cytotoxic drug.

In one embodiment, the anti-neoplastic agent comprises a compound selected from the group consisting of actinomycin-D, alkeran, ara-C, anastrozole, BiCNU, bicalutamide, bleomycin, busulfan, capecitabine, carboplatin, carboplatinum, carmustine, CCNU, chlorambucil, cisplatin, cladribine, CPT-11, cyclophosphamide, cytarabine, cytosine arabinoside, cytoxan, dacarbazine, dactinomycin, daunorubicin, dexrazoxane, docetaxel, doxorubicin, DTIC, epirubicin, ethyleneimine, etoposide, floxuridine, fludarabine, fluorouracil, flutamide, fotemustine, gemcitabine, hexamethylamine, hydroxyurea, idarubicin, ifosfamide, irinotecan, lomustine, mechlorethamine, melphalan, mercaptopurine, methotrexate, mitomycin, mitotane, mitoxantrone, oxaliplatin, paclitaxel, pamidronate, pentostatin, plicamycin, procarbazine, steroids, streptozocin, STI-571, tamoxifen, temozolomide, teniposide, tetrazine, thioguanine, thiotepa, tomudex, topotecan, treosulphan, trimetrexate, vinblastine, vincristine, vindesine, vinorelbine, VP-16, xeloda, asparaginase, AIN-457, bapineuzumab, belimumab, brentuximab, briakinumab, canakinumab, cetuximab, dalotuzumab, denosumab, epratuzumab, estafenatox, farletuzumab, figitumumab, galiximab, gemtuzumab, girentuximab (WX-G250), herceptin, ibritumomab, inotuzumab, ipilimumab, mepolizumab, muromonab-CD3, naptumomab, necitumumab, nimotuzumab, ocrelizumab, ofatumumab, otelixizumab, ozogamicin, pagibaximab, panitumumab, pertuzumab, ramucirumab, reslizumab, rituximab, REGN88, solanezumab, tanezumab, teplizumab, tiuxetan, tositumomab, trastuzumab, tremelimumab, vedolizumab, zalutumumab, zanolimumab, 5FC, accutane hoffmann-la roche, AEE788 novartis, AMG-102, anti neoplaston, AQ4N (Banoxantrone), AVANDIA (Rosiglitazone Maleate), avastin (Bevacizumab) genetech, BCNU, biCNU carmustine, CCI-779, CCNU, CCNU lomustine, celecoxib (Systemic), chloroquine, cilengitide (EMD 121974), CPT-11 (CAMPTOSAR, Irinotecan), dasatinib (BMS-354825, Sprycel), dendritic cell therapy, etoposide (Eposin, Etopophos, Vepesid), GDC-0449, gleevec (imatinib mesylate), gliadel wafer, hydroxychloroquine, IL-13, IMC-3G3, immune therapy, iressa (ZD-1839), lapatinib (GW572016), methotrexate for cancer (Systemic), novocure, OSI-774, PCV, RAD001 novartis (mTOR inhibitor), rapamycin (Rapamune, Sirolimus), RMP-7, RTA 744, simvastatin, sirolimus, sorafenib, SU-101, SU5416 sugen, sulfasalazine (Azulfidine), sutent (Pfizer), TARCEVA (erlotinib HCl), taxol, TEMODAR schering-plough, TGF-B anti-sense, thalomid (thalidomide), topotecan (Systemic), VEGF trap, VEGF-trap, vorinostat (SAHA), XL 765, XL184, XL765, zarnestra (tipifarnib), ZOCOR (simvastatin), cyclophosphamide (Cytoxan), (Alkeran), chlorambucil (Leukeran), thiopeta (Thioplex), busulfan (Myleran), procarbazine (Matulane), dacarbazine (DTIC), altretamine (Hexalen), clorambucil, cisplatin (Platinol), ifosafamide, methotrexate (MTX), 6-thiopurines (Mercaptopurine [6-MP], Thioguanine [6-TG]), mercaptopurine (Purinethol), fludarabine phosphate, (Leustatin), flurouracil (5-FU), cytarabine (ara-C), azacitidine, vinblastine (Velban), vincristine (Oncovin), podophyllotoxins (etoposide {VP-16} and teniposide {VM-26}), camptothecins (topotecan and irinotecan), taxanes such as paclitaxel (Taxol) and docetaxel (Taxotere), (Adriamycin, Rubex, Doxil), dactinomycin (Cosmegen), plicamycin (Mithramycin), mitomycin: (Mutamycin), bleomycin (Blenoxane), estrogen and androgen inhibitors (Tamoxifen), gonadotropin-releasing hormone agonists (Leuprolide and Goserelin (Zoladex)), aromatase inhibitors (Aminoglutethimide and Anastrozole (Arimidex)), amsacrine, asparaginase (El-spar), mitoxantrone (Novantrone), mitotane (Lysodren), retinoic acid derivatives, bone marrow growth factors (sargramostim and filgrastim), amifostine, pemetrexed, decitabine, iniparib, olaparib, veliparib, everolimus, vorinostat, entinostat (SNDX-275), mocetinostat (MGCD0103), panobinostat (LBH589), romidepsin, valproic acid, flavopiridol, olomoucine, roscovitine, kenpaullone, AG-024322 (Pfizer), fascaplysin, ryuvidine, purvalanol A, NU2058, BML-259, SU 9516, PD-0332991, P276-00, geldanamycin, tanespimycin, alvespimycin, radicicol, deguelin, BIIB021, cis-imidazoline, benzodiazepinedione, spiro-oxindoles, isoquinolinone, thiophene, 5-deazaflavin, tryptamine, aminopyridine, diaminopyrimidine, pyridoisoquinoline, pyrrolopyrazole, indolocarbazole, pyrrolopyrimidine, dianilinopyrimidine, benzamide, phthalazinone, tricyclic indole, benzimidazole, indazole, pyrrolocarbazole, isoindolinone, morpholinyl anthracycline, a maytansinoid, ducarmycin, auristatins, calicheamicins (DNA damaging agents), α-amanitin (RNA polymerase II inhibitor), centanamycin, pyrrolobenzodiazepine, streptonigtin, nitrogen mustards, nitrosorueas, alkane sulfonates, pyrimidine analogs, purine analogs, antimetabolites, folate analogs, anthracyclines, taxanes, vinca alkaloids, topoisomerase inhibitors, hormonal agents, or any combination thereof.

In another embodiment, the anti-neoplastic agent comprises a functional nucleic acid or a polynucleotide encoding a functional nucleic acid. In yet another embodiment, the functional nucleic acid inhibits a gene that promotes tumor cell proliferation, angiogenesis or resistance to chemotherapy and/or that inhibits apoptosis or cell cycle arrest. The functional nucleic acid can be, for example, siRNA, miRNA, shRNA, lincRNA, antisense RNA, or ribozyme.

In one embodiment, the anti-neoplastic agent comprises a polynucleotide encoding a gene that promotes apoptosis.

In a further embodiment, the minicell or killed cell has a diameter of about 100 nm to about 600 nm.

In one embodiment, the first surface component comprises a first polypeptide and the second surface component comprises a second polypeptide, wherein the first polypeptide and the second polypeptide share greater than 90% sequence identity with each other. In another embodiment, the first surface component comprises a first polypeptide and the second surface component comprises a second polypeptide, wherein the first polypeptide and the second polypeptide have less than 90% sequence identity with each other.

Also encompassed is a method of imaging a tumor in a subject comprising administering systemically to the subject a theranostic composition as described herein, wherein the composition comprises a diagnostically effective amount of a radio-imaging agent.

Also encompassed is a method for treating a tumor in a subject in need, comprising administering systemically to the subject a theranostic composition as described herein, wherein the composition comprises a therapeutically effective amount of a radio-imaging agent and a therapeutically effective amount of an anti-neoplastic agent.

In addition, encompassed is a method of imaging and treating a tumor in a subject in need, comprising administering systemically to the subject a theranostic composition as described herein, wherein the composition comprises a diagnostically effective amount of a radioimaging agent, wherein the amount of the radio-imaging agent is also therapeutically effective; and a therapeutically effective amount of an antineoplastic agent.

In addition encompassed is a method of adjusting the signal intensity of an imaged tumor in a subject comprising first systemically administering a first dose of a theranostic composition as described herein followed by imaging the tumor; second systemically administering a second dose of a theranostic composition as described herein followed by imaging the tumor, wherein (i) the second dose of a theranostic composition comprises a greater amount of the radio-imaging agent per minicell as compared to the first dose; or (ii) the second dose of a theranostic composition comprises a lesser amount of the radio-imaging agent per minicell as compared to the first dose; and then third comparing the imaging results following (a) and (b) to obtain the adjusted signal intensity.

In all of the methods described herein, the plurality of the purified, intact bacterially derived minicells can comprise at least about 10⁸ minicells.

In all of the methods described herein, the method can be used to diagnose and/or treat brain tumors or, alternatively, the tumor to be treated/diagnosed does not comprise a brain tumor.

While any tumor can be diagnosed and/or treated using the theranostic compositions of the invention, in one embodiment the tumor does not comprise a glioblastoma, astrocytic tumor, oligodendroglial tumor, ependymoma, craniopharyngioma, pituitary tumor, primary lymphoma of the brain, pineal gland tumor, primary germ cell tumor of the brain, or combination thereof. In another embodiment, the tumor does not comprise a spleen tumor or a liver tumor.

Finally, in all of the methods described herein, the subject can be a human.

Both the foregoing summary and the following description of the drawings and detailed description are exemplary and explanatory. They are intended to provide further details of the invention, but are not to be construed as limiting. Other objects, advantages, and novel features will be readily apparent to those skilled in the art from the following detailed description of the invention.

### BRIEF DESCRIPTION OF THE DRAWINGS

FIG. 1 is a schematic showing the composition of the minicell-based theranostic according to the present invention.

FIG. 2 is a schematic showing how the minicell-based theranostic according to the present invention delivers a cytotoxic drug and a radiolabel intracellularly into tumor cells and additionally the tumor microenvironment.

### DETAILED DESCRIPTION

The present invention is directed to a theranostic composition comprising a therapeutically effective dose of purified, intact bacterially derived minicells or killed bacterial cells, packaged with a therapeutically effective concentration or amount of an anti-neoplastic agent. In addition, a bispecific ligand is attached to a first surface component of the intact minicells, and the bispecific ligand has a first arm with a binding specificity for the minicell first surface component and a second arm carries binding specificity for a tumor cell surface receptor. In addition, a monospecific ligand is attached to a second surface component of the intact minicells. The monospecific ligand has at least one radio-imaging agent conjugated to the monospecific ligand at one or more conjugation residues of the monospecific ligand. Finally, the theranostic minicells can be coated with a polymer that reduces opsonisation via professional phagocytic cells. The bispecific ligand and the monospecific ligand can each be a polypeptide, a carbohydrate or a combination of a polypeptide and a carbohydrate.

Throughout this document, any reference to a “bacterial minicell” also encompasses “killed bacterial cells.”

The present invention is directed to new theranostic compositions and delivery systems utilizing the same. The theranostic compositions exhibit one or more of the following properties: (1) the theranostic avoids entry into normal tissues and hence prevents radiation damage to healthy cells, as well as avoids background noise in the imaging of a tumor; (2) entry of the delivery system is specific to the tumor microenvironment, which ensures that only the tumor is specifically imaged and irradiated; (3) the delivery system should be safe for intravenous administration in human cancer patients, and additionally multiple dosing should be possible without serious adverse events; (4) the theranostic should carry a potent radiolabel dose to ensure clear imaging of the tumor and at the same time deliver a potent therapeutic radiation dose to the tumor; (5) since many late stage tumors exhibit resistance to radiation-induced cell death, ideally, the theranostic should also carry a cytotoxic or super-cytotoxic drug load to overcome drug and apoptosis resistance in late stage cancers; (6) ideally, the theranostic should be engulfed by the tumor cells to ensure that the cytotoxic or super-cytotoxic drug is released intracellularly, and the drug should be able to bind to the target molecule to achieve cell death. Additionally, the release of the radiation intracellularly may enhance the therapeutic effect of the radiation that delivered to the tumor; (7) the theranostic should remain in circulation long enough to ensure that the theranostic enters into the tumor microenvironment to enable clear tumor imaging and therapeutic efficacy. Therefore, the theranostic should be able to avoid rapid opsonisation by professional phagocytic cells; and (8) the theranostic should have relative ease of manufacturing to ensure that most nuclear medicine laboratories can prepare the theranostic dose and administer into the patient in a timely manner, as most radiolabels have a short half-life.

In an exemplary embodiment, bacterial minicells are initially packaged with an anti-neoplastic agent, for example, a cytotoxic drug such as doxorubicin (other exemplary antineoplastic agents are described herein). A bispecific ligand, for example, a single chain bispecific antibody where one arm has binding specificity to the O-polysaccharide component of the lipopolysaccharide, which is a normal surface component of the minicells derived from Gram-negative bacteria, and the other arm can have specificity to a cancer cell surface exposed receptor e.g. Epidermal Growth Factor Receptor (EGFR) (other exemplary cancer or tumor cell surface receptors are described herein). The monospecific ligand can be a polypeptide with binding specificity to a minicell surface exposed protein or O-polysaccharide. A radio-imaging agent can be conjugated to this ligand.

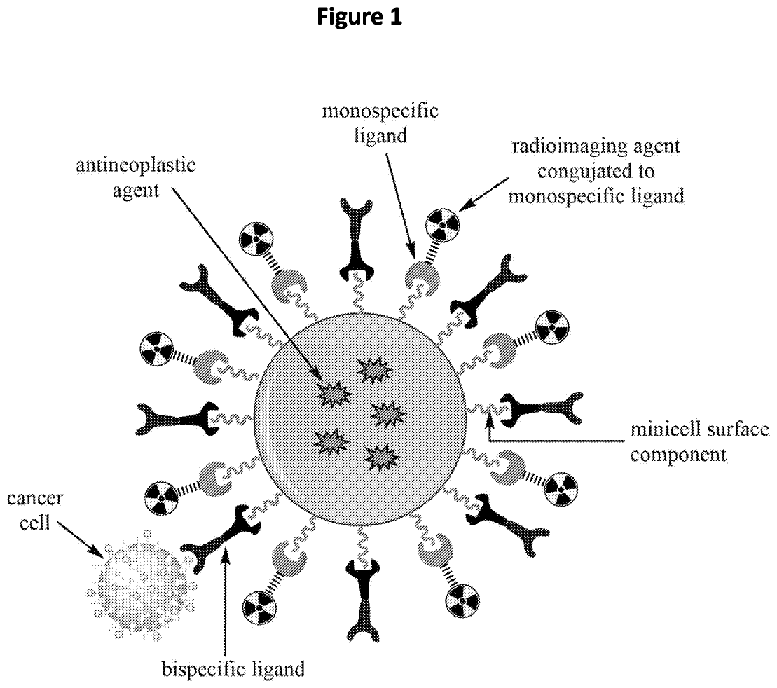

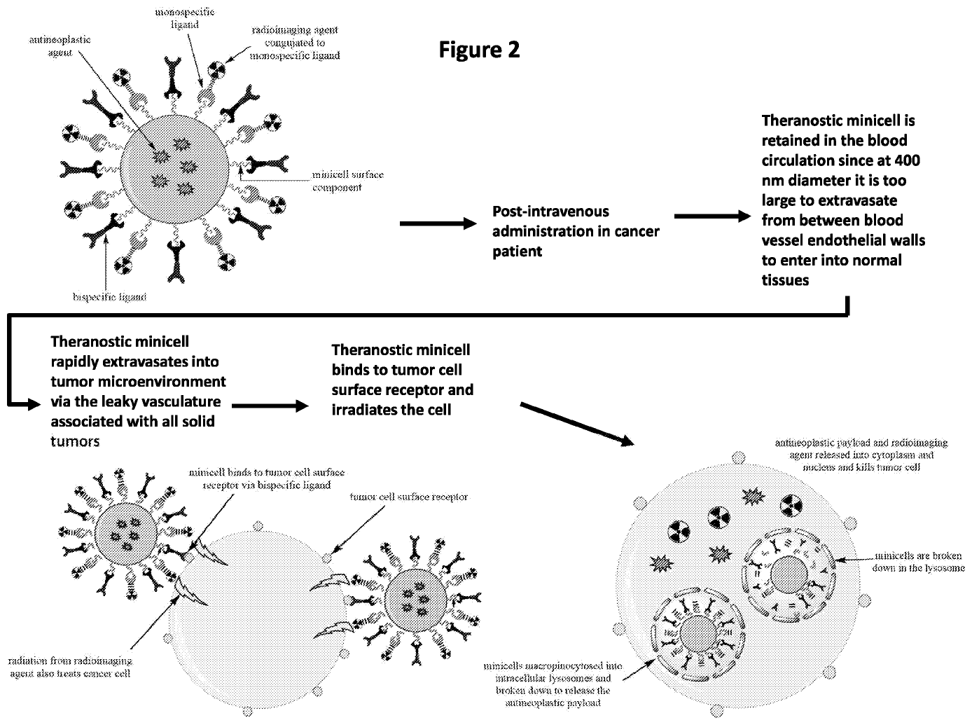

As shown in FIG. 1, the minicell surface now carries two types of ligands where one is the bispecific ligand providing specificity to attach to the tumor cell surface receptor and the other ligand carries a radio-imaging agent that is useful in imaging the minicells in the tumor but also irradiates the tumor cells when the minicells are bound via the tumor cell surface receptor. As shown in FIG. 2, post-receptor engagement, the minicells are macro-pinocytosed by the tumor cell, broken down in the intracellular lysosomes, and the cytotoxic drug is released intracellularly, thereby killing the tumor cells. Simultaneously, the radio-imaging agent is also released intracellularly, irradiating the tumor cell from within the cell and hence augmenting the tumor cell killing efficacy.

If the minicells are derived from Gram-positive or other bacteria that do not express lipopolysaccharide on the cell surface, then the bispecific and monospecific ligands can be designed to attach to minicell surface exposed proteins. Another way, and this applies to all bacterially derived minicells, is to genetically engineer the parent bacteria from which the minicells are derived so that these bacteria express hybrid outer membrane proteins with the ligand polypeptides exposed on the surface of the minicells. Such a design would not require the ligands to have specificity for the minicell surface component since they are integral parts of the outer membrane proteins and hence anchored on the minicell surface.

The method of preparing the minicells as described above, can also be carried out using killed bacteria instead of minicells. Therefore, here, the killed bacteria would carry the bispecific ligand and the monospecific ligand conjugated to the radio-imaging agent on its surface.

Currently, there is no single theranostic that satisfies the above needs. Surprisingly, the present invention satisfies all of these needs.

### I. Composition Components

A. Minicells

“Minicells” refer to a derivative of a bacterial cell that is lacking in chromosomes (“chromosome-free”) and is engendered by a disturbance in the coordination, during binary fission, of cell division with DNA segregation. Minicells are distinct from other small vesicles, such as so-called “membrane blebs” (˜0.2 μm or less in size), which are generated and released spontaneously in certain situations but which are not due to specific genetic rearrangements or episomal gene expression. By the same token, intact minicells are distinct from bacterial ghosts, which are not generated due to specific genetic rearrangements or episomal gene expression. Bacterially derived minicells employed in this disclosure are fully intact and, thus, are distinguished from other chromosome-free forms of bacterial cellular derivatives characterized by an outer or defining membrane that is disrupted or degraded, even removed. See e.g., U.S. Pat. No. 7,183,105 at col. 111, lines 54 et seq. The intact membrane that characterizes the minicells of the present disclosure allows retention of the therapeutic (anti-neoplastic agent) payload within the minicell until the payload is released, post-uptake, within a tumor cell.

The minicells employed in this disclosure can be prepared from bacterial cells, such as but not limited to E. coli and S. typhimurium. Prokaryotic chromosomal replication is linked to normal binary fission, which involves mid-cell septum formation. In E. coli, for example, mutation of min genes, such as minCD, can remove the inhibition of septum formation at the cell poles during cell division, resulting in production of a normal daughter cell and a chromosome-less minicell. de Boer et al., 1992; Raskin & de Boer, 1999; Hu & Lutkenhaus, 1999; Harry, 2001.

In addition to min operon mutations, chromosome-less minicells also are generated following a range of other genetic rearrangements or mutations that affect septum formation, for example, in the divIVB1 in B. subtilis. Reeve and Cornett (1975). Minicells also can be formed following a perturbation in the levels of gene expression of proteins involved in cell division/chromosome segregation. For instance, over-expression of minE leads to polar division and production of minicells. Similarly, chromosome-less minicells can result from defects in chromosome segregation, e.g., the smc mutation in Bacillus subtilis [Britton et al., 1998], the spoOJ deletion in B. subtilis [Ireton et al., 1994], the mukB mutation in E. coli [Hiraga et al., 1989], and the parC mutation in E. coli [Stewart and D'Ari, 1992]. Further, CafA can enhance the rate of cell division and/or inhibit chromosome partitioning after replication [Okada et al., 1994], resulting in formation of chained cells and chromosome-less minicells.

Accordingly, minicells can be prepared for the present disclosure from any bacterial cell, be it of Gram-positive or Gram-negative origin. Furthermore, the minicells used in the disclosure should possess intact cell walls (i.e., are “intact minicells”), as noted above, and should be distinguished over and separated from other small vesicles, such as membrane blebs, which are not attributable to specific genetic rearrangements or episomal gene expression.

In a given embodiment, the parental (source) bacteria for the minicells can be Gram positive, or they can be Gram negative, as mentioned. In one aspect, therefore, the parental bacteria are one or more selected from Terra-/Glidobacteria (BV1), Proteobacteria (BV2), BV4 including Spirochaetes, Sphingobacteria, and Planctobacteria. Pursuant to another aspect, the bacteria are one or more selected from Firmicutes (BV3) such as Bacilli, Clostridia or Tenericutes/Mollicutes, or Actinobacteria (BV5) such as Actinomycetales or Bifidobacteriales.

In yet a further aspect, the bacteria are one or more selected from Eobacteria (Chloroflexi, Deinococcus-Thermus), Cyanobacteria, Thermodesulfobacteria, thermophiles (Aquificae, Thermotogae), Alpha, Beta, Gamma (Enterobacteriaceae), Delta or Epsilon Proteobacteria, Spirochaetes, Fibrobacteres, Chlorobi/Bacteroidetes, Chlamydiae/Verrucomicrobia, Planctomycetes, Acidobacteria, Chrysiogenetes, Deferribacteres, Fusobacteria, Gemmatimonadetes, Nitrospirae, Synergistetes, Dictyoglomi, Lentisphaerae Bacillales, Bacillaceae, Listeriaceae, Staphylococcaceae, Lactobacillales, Enterococcaceae, Lactobacillaceae, Leuconostocaceae, Streptococcaceae, Clostridiales, Halanaerobiales, Thermoanaerobacterales, Mycoplasmatales, Entomoplasmatales, Anaeroplasmatales, Acholeplasmatales, Haloplasmatales, Actinomycineae, Actinomycetaceae, Corynebacterineae, Mycobacteriaceae, Nocardiaceae, Corynebacteriaceae, Frankineae, Frankiaceae, Micrococcineae, Brevibacteriaceae, and Bifidobacteriaceae.

For pharmaceutical use, a theranostic composition of the disclosure should comprise minicells that are isolated as thoroughly as possible from immunogenic components and other toxic contaminants. Methodology for purifying bacterially derived minicells to remove free endotoxin and parent bacterial cells are described in WO 2004/113507, which is incorporated by reference here in its entirety. Briefly, the purification process achieves removal of (a) smaller vesicles, such as membrane blebs, which are generally smaller than 0.2 μm in size, (b) free endotoxins released from cell membranes, and (c) parental bacteria, whether live or dead, and their debris, which are sources of free endotoxins, too. Such removal can be implemented with, inter alia, a 0.2 μm filter to remove smaller vesicles and cell debris, a 0.45 μm filter to remove parental cells following induction of the parental cells to form filaments, antibiotics to kill live bacterial cells, and antibodies against free endotoxins.

Underlying the purification procedure is a discovery by the present inventors that, despite the difference of their bacterial sources, all intact minicells are approximately 400 nm in size, i.e., larger than membrane blebs and other smaller vesicles and yet smaller than parental bacteria. Size determination for minicells can be accomplished by using solid-state, such as electron microscopy, or by liquid-based techniques, e.g., dynamic light scattering. The size value yielded by each such technique can have an error range, and the values can differ somewhat between techniques. Thus, the size of minicells in a dried state can be measured via electron microscopy as approximately 400 nm±50 nm. On the other hand, dynamic light scattering can measure the same minicells to be approximately 500 nm±50 nm in size. Also, drug-packaged, ligand-targeted minicells can be measured, again using dynamic light scattering, to be approximately 600 nm±50 nm.

This scatter of size values is readily accommodated in practice, e.g., for purposes of isolating minicells from immunogenic components and other toxic contaminants, as described above. That is, an intact, bacterially derived minicell is characterized by cytoplasm surrounded by a rigid membrane, which gives the minicell a rigid, spherical structure. This structure is evident in transmission-electron micrographs, in which minicell diameter is measured, across the minicell, between the outer limits of the rigid membrane. This measurement provides the above-mentioned size value of 400 nm±50 nm.

Another structural element of a minicell derived from Gram-negative bacteria is the O-polysaccharide component of lipopolysaccharide (LPS), which is embedded in the outer membrane via the lipid A anchor. The component is a chain of repeat carbohydrate-residue units, with as many as 70 to 100 repeat units of four to five sugars per chain. Because these chains are not rigid, in a liquid environment, as in vivo, they can adopt a waving, flexible structure that gives the general appearance of seaweed in a coral sea environment; i.e., the chains move with the liquid while remaining anchored to the minicell membrane.

Influenced by the O-polysaccharide component, dynamic light scattering can provide a value for minicell size of about 500 nm to about 600 nm, as noted above. Nevertheless, minicells from Gram-negative and Gram-positive bacteria alike readily pass through a 0.45 μm filter, which substantiates an effective minicell size of 400 nm±50 nm. The above-mentioned scatter in sizes is encompassed by the present invention and, in particular, is denoted by the qualifier “approximately” in the phrase “approximately 400 nm in size” and the like.

In relation to toxic contaminants, a composition of the disclosure can contain less than about 350 EU free endotoxin. Illustrative in this regard are levels of free endotoxin of about 250 EU, about 200 EU, about 150 EU, about 100 EU, about 90 EU, about 80 EU, about 70 EU, about 60 EU, about 50 EU, about 40 EU, about 30 EU, about 20 EU, about 15 EU, about 10 EU, about 9 EU, about 8 EU, about 7 EU, about 6 EU, about 5 EU, about 4 EU, about 3 EU, about 2 EU, about 1 EU, about 0.9 EU, about 0.8 EU, about 0.7 EU, about 0.6 EU, about 0.5 EU, about 0.4 EU, about 0.3 EU, about 0.2 EU, about 0.1 EU, about 0.05 EU, and about 0.01 EU, respectively.

A theranostic composition of the disclosure also can contain at least about 10⁸ minicells, e.g., at least about 5×10⁸. Alternatively, the composition can contain on the order of about 10⁹ or about 10¹⁰ minicells, e.g., about 5×10⁹, about 1×10¹⁰ or about 5×10¹⁰ minicells. Amongst any such number of minicells, moreover, a composition of the disclosure can contain fewer than about 10 contaminating parent bacterial cells, e.g., fewer than about 9, about 8, about 7, about 6, about 5, about 4, about 3, about 2, or about 1 parent bacterial cells.

B. Tumor Cell Surface Receptors

Tumor cell surface receptors can be selected from a number of known receptors. The bispecific ligand for the present invention carries a tumor cell-surface binding site at one end of the ligand. A number of tumor cell surface receptors have been identified as described below. Peptides that specifically bind to these receptors have also been identified, again as described below. The present invention allows the bispecific ligand, which binds both the minicell first surface component and the tumor cell surface receptor, to hold the minicell, its anti-neoplastic agent, and the radio-imaging agent conjugated to the monospecific ligand, in close proximity to the tumor cell, delivering a concentrated dose of radiation to the targeted cancer cell, while sparing healthy cells. Furthermore, the minicell is held proximal to the tumor cell to facilitate endocytosis and ultimately release of anti-neoplastic cargo and radionuclide within the tumor cell to further treat the tumor.

In some embodiments, the tumor cell surface receptor comprises an integrin, neuromedin B receptor, bombesin 3 receptor, GRP receptor, bombesin 4 receptor, CCK2/gastrin, melanocortin-1 receptor (MC-1r), neuropeptide Y (NPY) receptor, neutrotensin (NT) receptor, prostate specific membrane antigen (PSMA), somatostatin (SST) receptor, neurokinin 1 receptor (NK1R), chemokine receptor type 4 (CXCR4), vasoactive intestinal peptide (VIP), epidermal growth factor receptor (EGFR), vascular endothelial growth factor receptor (VEGFR), platelet-derived growth factor receptor (PDGFR), insulin-like growth factor receptor (IGFR), or any combination thereof.

In another embodiment, the tumor cell surface receptor comprises EpCAM, CCR5, CD19, HER-2 neu, HER-3, HER-4, EGFR, PSMA, CEA, MUC-1 (mucin), MUC2, MUC3, MUC4, MUC5, MUC5, MUC7, BhcG, Lewis-Y, CD20, CD33, CD30, ganglioside GD3, 9-O-Acetyl-GD3, GM2, Globo H, fucosyl GM1, Poly SA, GD2, carboanhydrase IX (MN/CA IX), CD44v6, sonic hedgehog (Shh), Wue-1, Plasma Cell Antigen, (membrane-bound) IgE, melanoma chondroitin sulfate proteoglycan (MCSP), CCR8, TNF-alpha precursor, STEAP, mesothelin, A33 antigen, prostate stem cell antigen (PSCA), Ly-6; desmoglein 4, E-cadherin neoepitope, fetal acetylcholine receptor, CD25, CA19-9 marker, CA-125 marker and muellerian inhibitory substance (MIS) receptor type II, sTn (sialylated Tn antigen; TAG-72), FAP (fibroblast activation antigen), endosialin, EGFRVIII, LG, SAS, CD63, or any combination thereof.

C. Monospecific Ligand and Radio-Imaging Agent

The present invention includes a monospecific ligand which binds to a minicell second surface component. The monospecific ligand is also conjugated to a radio-imaging agent. The monospecific ligand may include a peptide, which may be a known peptide. These peptides have been conjugated with various radiolabels for use of the radiolabeled peptide as an in-vivo imaging agent of tumor cells, where the minicell targets the imaged tumor cell by way of the bispecific ligand that binds the tumor cell surface receptor.

In some embodiments, the radio-imaging agent is conjugated to the monospecific ligand via a linker. In some embodiments, the amount of radio-imaging agent conjugated to the monospecific ligand varies directly with the amount of conjugation residues. In some embodiments, the monospecific ligand can be increased in length to produce a corresponding increase in an amount of conjugation residues and radio-imaging agent. In some embodiments, the one or more conjugation residues is selected from the group consisting of an F-amino group on a lysine side chain, a guanidinium group on an arginine side chain, a carboxyl group on an aspartic acid or glutamic acid, a cysteine thiol, a phenol on a tyrosine, and a combination thereof.

In some embodiments, the monospecific ligand comprises a polypeptide, aptamer, carbohydrate, or combination thereof. In some embodiments, the monospecific ligand comprises a polypeptide of variable length comprising from about 15 to about 500 amino acids. In other embodiments, the monospecific ligand comprises a polypeptide of variable length comprising from about 15, about 25, about 50, about 75, about 100, about 125, about 150, about 175, about 200, about 225, about 250, about 275, about 300, about 325, about 350, about 375, about 400, about 425, about 450, about 475, or about 500 amino acids, or any number of amino acids in-between the values of about 15 to about 500. In some embodiments, the monospecific ligand comprises a polypeptide of variable length comprising from about 10 to about 400 conjugation residues. In other embodiments, the monospecific ligand comprises a polypeptide of variable length comprising from about 10, about 25, about 50, about 75, about 100, about 125, about 150, about 175, about 200, about 225, about 250, about 275, about 300, about 325, about 350, about 375, or about 400 conjugation residues, or any number of conjugation residues in-between the values of about 10 to about 400.

D. Imaging Moiety

In some embodiments, the radioimaging agent comprises a radioisotope, magnetic nanoparticle, organic fluorescent dye, or any combination thereof. The imaging moiety such as radioisotope for positron emission tomography (PET) or single photon emission computed tomography (SPECT), magnetic nanoparticle for magnetic resonance imaging (MRI), or organic fluorescent dye for optical imaging is conjugated to the bispecific ligand and this conjugation can be done via a linker. In some embodiments, the theranostic composition comprises a quantity of radio-imaging agent sufficient to produce a clear image of the tumor upon radioimaging. Such conjugation procedures between the imaging moiety and a ligand, such as a peptide, aptamer, protein or antibody has been described previously (Chen et al., 2010; James et al., 2012; Lin et al., 2015). Here, such a ligand may include the monospecific binding ligand, conjugated to the imaging moiety and bound to the minicell.

The minicells of the present invention, targeted to the tumor cells will also deliver targeted radiation from the radioimaging agent to the tumor cell to which the minicell is bound. In some embodiments, the radio-imaging agent also functions as a therapeutic radiation emitting agent, and wherein the amount of radiation provided by the radio-imaging agent is sufficient to provide a therapeutic effect on the tumor. In some embodiments, the therapeutic effect is a reduction in tumor size. The tumor may be reduced in size by about 300%, about 275%, about 250%, about 225%, about 200%, about 175%, about 150%, about 125%, about 100%, about 90%, about 80%, about 70%, about 60%, about 50%, about 40%, about 30%, about 20%, about 10%, or about 5%. The radiation level of each minicell may be about 30 Gy to about 100 Gy radiation, or any amount inbetween these two values.

The functional groups of peptides available for conjugation include but are not limited to the F-amino group on lysine side chains, the guanidinium group on arginine side chains, the carboxyl groups on aspartic acid or glutamic acid, the cysteine thiol, and the phenol on tyrosine.

The most common conjugation reactions are carbodiimide/N-hydroxysuccinimidyl (EDC/NHS) mediated carboxyl and amine coupling, maleimide conjugation to thiol groups, and diazonium modification of the phenol on tyrosine. The representative chemistries to couple peptides with imaging moieties can be found in a number of reviews [Erathodiyil and Ying, 2011; Takahashi et al., 2008].

To develop minicell based theranostics, the radionuclides should be labeled onto the monospecific ligand described herein. Several radionuclides have been used for peptide labeling including ^{99m}Tc, ¹²³I, and ¹¹¹In for SPECT imaging and ¹⁸F, ⁶⁴Cu and ⁶⁸Ga for PET imaging [Chatalic et al., 2015]. Generally, these radionuclides are attached to the peptides via chelators. Some widely-used chelators are described in [Sun et al., 2017].

Radiolabels: Radiolabels useful for attaching to minicells for theranostic purposes include, for example, Iodine-131 and lutetium-177, which are gamma and beta emitters. Thus, these agents can be used for both imaging and therapy.

Different isotopes of the same element, for example, iodine-123 (gamma emitter) and iodine-131 (gamma and beta emitters), can also be used for theranostic purposes [Gerard and Cavalieri, 2002; Alzahrani et al., 2012].

Newer examples are yttrium-86/yttrium-90 or terbium isotopes (Tb): ¹⁵²Tb (beta plus emitter), ¹⁵⁵Tb (gamma emitter), ¹⁴⁹Tb (alpha emitter), and ¹⁶¹Tb (beta minus particle) [Müller et al., 2012; Walrand et al., 2015].

Nuclear imaging utilizes gamma and positron emitters (β+). Gamma emitters, such as technetium-99m (^{99m}Tc) or iodine-123 (¹²³I), can be located using gamma cameras (planar imaging) or SPECT (single photon emission computed tomography) [Holman and Tumeh, 1990].

Better resolution can be achieved via PET (positron emission tomography) using positron emitters, such as gallium-68 (⁶⁸Ga) and fluor-18 (¹⁸F) [Eckelman and Gibson, 1993]. Most therapeutic radiopharmaceuticals are labeled with beta-emitting isotopes (β-).

The tissue penetration of these particles is proportional to the energy of the radioisotopes [Kramer-Marek and Capala, 2012]. Beta particles have a potential cytocidal effect, but they also spare the surrounding healthy tissue due to having a tissue penetration of only a few millimeters. Commonly used beta emitters in routine nuclear oncology practices include lutetium-177 (¹⁷⁷Lu, tissue penetration: about 0.5- about 0.6 mm, maximum: about 2 mm, 497 keV, half-life: 6.7 days) and yttrium-90 (⁹⁰Y, tissue penetration: mean 2.5 mm, maximum: about 11 mm, 935 keV, half-life: 64 hours) [Teunissen et al., 2005; Kwekkeboom et al., 2008; Ahmadzadehfar et al., 2010; Pillai et al., 2013; Ahmadzadehfar et al., 2016].

Radiolabeled phosphonates have a high bone affinity and can be used for imaging and palliation of painful bone metastases. Depending on the degree of osseous metabolism, the tracer accumulates via adhesion to bones and, preferably, to osteoblastic bone metastases. Therapy planning requires a bone scintigraphy with technetium-99m-hydroxyethylidene diphosphonate (HEDP) to estimate the metabolism and the extent of the metastases involvement. Bisphosphonate HEDP can be labeled for therapy either with rhenium-186 (beta-emitter, half-life: 89 hours, 1.1 MeV maximal energy, maximal range: 4.6 mm) or rhenium-188 (beta-emitter [to 85%, 2.1 MeV] and gamma-emitter [to 15%,155 keV], half-life: 16.8 hours, maximal range in soft tissue: 10 mm) [Palmedo, 2007]. New promising radiopharmaceuticals for bone palliation therapy include radiolabeled complexes of zoledronic acid. Zoledronic acid belongs to a new, most potent generation of bisphosphonates with cyclic side chains. The bone affinity of zoledronic acid labeled with scandium-46 or lutetium-177 has shown excellent absorption (98% for [¹⁷⁷Lu]Lu-zoledronate and 82% for [⁴⁶Sc]Sc-zoledronate), which is much higher than of bisphosphonates labeled with samarium-153 (maximum: 67%) [Majkowska et al., 2009]. These bisphosphonates can be conjugated to intact minicells for use as theranostics for bone metastasis.

Thus, in some embodiments, the radio-imaging agent comprises a radioisotope selected from the group consisting of yttrium-90, yttrium-86, terbium-152, terbium-155, terbium-149, terbium-161, technetium-99m, iodine-123, iodine-131, rubidium-82, thallium-201, gallium-67, fluorine-18, copper-64, gallium-68, xenon-133, indium-111, lutetium-177, and any combination thereof.

E. Tumor Targeting Ligands & Antigens

A number of tumor targeting ligands are known in the art (Hong et al., 2011; Hoelder et al., 2012; Galluzzi et al., 2013).

Several peptides, such as somatostatin (SST) peptide, vasoactive intestinal peptide (VIP), Arg-Gly-Asp (RGD) peptide, and bombesin/gastrin-releasing peptide (BBN/GRP), have been successfully characterized for tumor receptor imaging [De Jong et al., 2009; Tweedle, 2009; Schottelius and Wester 2009; Igarashi et al., 2011; Laverman et al., 2012].

Tumor-targeting peptide sequences can be selected mainly in three different ways: (1) derivatization from natural proteins (Nagpal et al., 2011); (2) chemical synthesis and structure-based rational engineering (Andersson et al., 2000; Merrifield, 2006); and (3) screening of peptide libraries (Gray and Brown 2013). Among the methods, phage display technology is a conventional but most widely used method with many advantages such as ease of handling and large numbers of different peptides can be screened effectively [Deutscher, 2010].

Representative peptides for in vivo imaging: Receptors that are overexpressed on tumor cells rather than on normal cells are excellent candidates for in vivo tumor imaging. To date, many tumor targeting peptides and their analogs have been identified as described below.

Arg-Gly-Asp (RGD) peptide-RGD specifically binds to integrin receptors [Ruoslahti, 1996]. Integrins constitute two subunits (α and β subunits). The integrin family, especially αvβ₃, is associated with tumor angiogenesis and metastasis. They are overexpressed on endothelial cells during angiogenesis, but barely detectable in most normal organs. Therefore, they are widely used for diagnostic imaging.

Bombesin (BBN)/gastrin-releasing peptide (GRP)—Amphibian BBNs and their related peptides consist of a family of neuropeptides exhibiting various physiological effects such as exocrine and endocrine secretions, thermoregulation, sucrose regulations as well as cell growth [Ohki-Hamazaki et al., 2005]. The bombesin-like peptide receptors have 4-subtypes: the neuromedin B receptor, the bombesin 3 receptor, the GRP receptor, and the bombesin 4 receptor. These receptors are overexpressed in many tumors such as breast cancer, ovarian cancer and gastrointestinal stromal tumors.

Cholecystokinin (CCK)/gastrin peptide—CCK and gastrin are structurally and functionally similar peptides that exert a variety of physiological actions in the gastrointestinal tract as well as the central nervous system [Matsuno et al., 1997]. Three types of receptors for CCK (CCK1, CCK2 and CCK2i4sv have been identified, which all belong to the superfamily of GPCRs. Among them, CCK2/gastrin receptors have been frequently found in human cancers such as stromal ovarian cancers and astrocytomas.

α-Melanocyte-stimulating hormone (α-MSH)—α-MSHs are linear tridecapeptides, mainly responsible for skin pigmentation regulation [Singh and Mukhopadhyay, 2014]. α-MSHs and their analogs exhibit binding affinities to melanocortin-1 receptors (MC-1r) which are expressed in over 80% of human melanoma metastases, and thus, are widely used as vehicles for melanoma-targeted imaging and radiotherapy.

Neuropeptide Y (NPY)—NPY is a 36 amino acid peptide and belongs to the pancreatic polypeptide family [Tatemoto, 2004]. NPY receptors are overexpressed in various tumors including neuroblastomas, sarcomas, and breast cancers.

Neurotensin (NT)—NT is a 13 amino acid peptide, targeting NT receptor which has been identified in various tumors such as ductal pancreatic adenocarcinomas, small cell lung cancer, and medullary thyroid cancer [Tyler-McMahon et al., 2000]. Therefore, it is an attractive candidate for cancer imaging.

Prostate Specific Membrane Antigen (PSMA)—Prostate cancer cells overexpress PSMA on the cell surface [Silver et al., 2007; Ghosh and Heston, 2004; Mhawech-Fauceglia et al., 2007; Santoni et al., 2014]. There are several available radiopharmaceuticals that target PSMA including [⁶⁸Ga]Ga-PSMA-HBED-CC (also known as [⁶⁸Ga]Ga-PSMA-11 [PET]), a monoclonal antibody (mAb) [¹⁷⁷Lu]Lu/[⁹⁰Y]Y-J591 (therapy), [¹²³I]I-MIP-1072 (planar/SPECT), [¹³¹I]I-MIP-1095 (therapy), and the theranostic agents PSMA-I&T and DKFZ-PSMA-617 (PSMA-617), which are labeled with ⁶⁸Ga for PET or with ¹⁷⁷Lu for therapy.

Somatostatin (SST) peptide—SSTs are naturally occurring cyclopeptide hormones with either 14 or 28 amino acids [Weckbecker et al., 2003]. They can inhibit the secretion of insulin, glucagon and some other hormones. Somatostatin receptors (SSTRs; five subtypes SSTR1-SSTR5) are overexpressed in many tumors including gliomas, neuroendocrine tumors and breast tumor. Neuroendocrine neoplasia (NEN) of the GEP system originates most frequently from the pancreas, jejunum, ileum, cecum, rectum, appendix, and colon. The common characteristic of all GEP-NEN is the compound features of endocrine and nerve cells. Well-differentiated NEN overexpresses somatostatin receptors (SSTRs), especially the SSTR-2 subtype.

Substance P—Substance P is an undecapeptide belonging to a family of neuropeptides known as tachykinins [Strand, 1999]. Substance P is a specific endogenous ligand known for neurokinin 1 receptor (NKiR) which is found to be expressed on various cancer cells.

T140—T140 is a 14 amino acid peptide with one disulfide bridge and is an inverse agonist of chemokine receptor type 4 (CXCR4) [Burger et al., 2005]. Its derivatives are widely used as CXCR4 imaging agents.

Tumor molecular targeted peptide 1 (TMTP1)—TMTP1 is a 5-amino acid peptide that has been found to specifically bind to highly metastatic cancer cells, especially those from a typical liver micrometastasis [Yang et al., 2008].

Vasoactive intestinal peptide (VIP)—VIP is a neuropeptide with 28 amino acids [Igarashi et al., 2011]. It promotes vasodilation, cell growth and proliferation. Its action is mainly controlled by two receptor subtypes (VPAC1 and VPAC2). A large amount of VIP receptors are expressed on many tumors including adenocarcinomas of the pancreas and neuroendocrine tumors.

Thus, in some embodiments, the bispecific ligand or the second arm of the bispecific ligand comprises Arg-Gly-Asp (RGD) peptide, bombesin (BBN)/gastrin-releasing peptide (GRP), cholecystokinin (CCK)/gastrin peptide, α-melanocyte-stimulating hormone (α-MSH), neuropeptide Y (NPY), neutrotensin (NT), [⁶⁸Ga]Ga PSMA HBED CC ([⁶⁸Ga]Ga-PSMA-11 [PET]), [¹⁷⁷Lu]Lu/[⁹⁰Y]Y-J591, [¹²³I]I-MIP-1072, [¹³¹I]I-MIP-1095, ⁶⁸Ga or ¹⁷⁷Lu labeled PSMA-I&T, 68Ga or ¹⁷⁷Lu labeled DKFZ-PSMA-617 (PSMA-617), somatostatin (SST) peptide, substance P, T140, tumor molecular targeted peptide 1 (TMTP1), vasoactive intestinal peptide (VIP), or any combination thereof.

In one embodiment of the invention, the tumor cell surface receptor comprises an integrin, neuromedin B receptor, bombesin 3 receptor, GRP receptor, bombesin 4 receptor, CCK2/gastrin, melanocortin-1 receptor (MC-1r), neuropeptide Y (NPY) receptor, neutrotensin (NT) receptor, prostate specific membrane antigen (PSMA), somatostatin (SST) receptor, neurokinin 1 receptor (NK1R), chemokine receptor type 4 (CXCR4), vasoactive intestinal peptide (VIP), epidermal growth factor receptor (EGFR), vascular endothelial growth factor receptor (VEGFR), platelet-derived growth factor receptor (PDGFR), insulin-like growth factor receptor (IGFR), or any combination thereof.

According to another embodiment of the invention, the target antigen is an antigen which is uniquely expressed on a target cell in a disease condition, but which remains either non-expressed, expressed at a low level or non-accessible in a healthy condition. Examples of such target antigens which might be specifically bound by a bispecific antibody of the invention may advantageously be selected from EpCAM, CCR5, CD19, HER-2 neu, HER-3, HER-4, EGFR, PSMA, CEA, MUC-1 (mucin), MUC2, MUC3, MUC4, MUC5, MUC5, MUC7, BhcG, Lewis-Y. CD20, CD33, CD30, ganglioside GD3, 9-O-Acetyl-GD3, GM2, Globo H, fucosyl GM1, Poly SA, GD2, Carboanhydrase IX (MN/CA IX), CD44v6, Sonic Hedgehog (Shh), Wue-1, Plasma Cell Antigen, (membrane-bound) IgE, Melanoma Chondroitin Sulfate Proteoglycan (MCSP), CCR8, TNF-alpha precursor, STEAP, mesothelin, A33 Antigen, Prostate Stem Cell Antigen (PSCA), Ly-6; desmoglein 4, E-cadherin neoepitope, Fetal Acetylcholine Receptor, CD25, CA19-9 marker, CA-125 marker and Muellerian Inhibitory Substance (MIS) Receptor type II, sTn (sialylated Tn antigen; TAG-72), FAP (fibroblast activation antigen), endosialin, EGFRVIII, LG, SAS and CD63.

F. Antineoplastic Agents

In the context of this disclosure, selecting an anti-neoplastic agent for treating a given tumor patient depends on several factors, in keeping with conventional medical practice. These factors include but are not limited to the patient's age, Karnofsky Score, and whatever previous therapy the patient may have received. See, generally, PRINCIPLES AND PRACTICE OF NEURO-ONCOLOGY, M. Mehta (Demos Medical Publishing 2011), and PRINCIPLES OF NEURO-ONCOLOGY, D. Schiff and P. O'Neill, eds. (McGraw-Hill 2005).

In one exemplary embodiment, a glycolipid such as α-galactosyl ceramide is present as a payload packaged within a bacterial minicell. Bacterial minicells carrying the glycolipid, once broken down in a lysosome, deliver the glycolipid intact to the cell.

In accordance with the disclosure, an antineoplastic agent can be selected from one of the classes detailed below, for packaging into intact, bacterially derived minicells, which then are administered to treat a cancer.
Polyfunctional alkylating agents, exemplified by Cyclophosphamide (Cytoxan), Mechlorethamine, Melphalan (Alkeran), Chlorambucil (Leukeran), Thiopeta (Thioplex), Busulfan (Myleran).
Alkylating drugs, exemplified by Procarbazine (Matulane), Dacarbazine (DTIC), Altretamine (Hexalen), Clorambucil, Cisplatin (Platinol), Carboplatin, Ifosafamide, Oxaliplatin.
Antimetabolites, exemplified by Methotrexate (MTX), 6-Thiopurines (Mercaptopurine [6-MP], Thioguanine [6-TG]), Mercaptopurine (Purinethol), Thioguanine, Fludarabine phosphate, Cladribine: (Leustatin), Pentostatin, Flurouracil (5-FU), Cytarabine (ara-C), Azacitidine.
Plant alkaloids, terpenoids and topoisomerase inhibitors, exemplified by Vinblastine (Velban), Vincristine (Oncovin), Vindesine, Vinorelbine, Podophyllotoxins (etoposide {VP-16} and teniposide {VM-26}), Camptothecins (topotecan and irinotecan), Taxanes such as Paclitaxel (Taxol) and Docetaxel (Taxotere).
Antibiotics, exemplified by Doxorubicin (Adriamycin, Rubex, Doxil), Daunorubicin, Idarubicin, Dactinomycin (Cosmegen), Plicamycin (Mithramycin), Mitomycin: (Mutamycin), Bleomycin (Blenoxane).
Hormonal agents, exemplified by Estrogen and Androgen Inhibitors (Tamoxifen and Flutamide), Gonadotropin-Releasing Hormone Agonists (Leuprolide and Goserelin (Zoladex)), Aromatase Inhibitors (Aminoglutethimide and Anastrozole (Arimidex)).
Miscellaneous Anticancer Drugs, exemplified by Amsacrine, Asparaginase (El-spar), Hydroxyurea, Mitoxantrone (Novantrone), Mitotane (Lysodren), Retinoic acid Derivatives, Bone Marrow Growth Factors (sargramostim and filgrastim), Amifostine.
Agents disrupting folate metabolism, e.g., Pemetrexed.
DNA hypomethylating agents, e.g., Azacitidine, Decitabine.
Poly(adenosine diphosphate [ADP]-ribose) polymerase (PARP) pathway inhibitors, such as Iniparib, Olaparib, Veliparib.
PI3K/Akt/mTOR pathway inhibitors, e.g., Everolimus.
Histone deacetylase (HDAC) inhibitors, e.g., Vorinostat, Entinostat (SNDX-275), Mocetinostat (MGCD0103), Panobinostat (LBH589), Romidepsin, Valproic acid.
Cyclin-dependent kinase (CDK) inhibitors, e.g., Flavopiridol, Olomoucine, Roscovitine, Kenpaullone, AG-024322 (Pfizer), Fascaplysin, Ryuvidine, Purvalanol A, NU2058, BML-259, SU 9516, PD-0332991, P276-00.
Heat shock protein (HSP90) inhibitors, e.g., Geldanamycin, Tanespimycin, Alvespimycin, Radicicol, Deguelin, BIIB021.
Murine double minute 2 (MDM2) inhibitors, e.g., Cis-imidazoline, Benzodiazepinedione, Spiro-oxindoles, Isoquinolinone, Thiophene, 5-Deazaflavin, Tryptamine.
Anaplastic lymphoma kinase (ALK) inhibitors, e.g., Aminopyridine, Diaminopyrimidine, Pyridoisoquinoline, Pyrrolopyrazole, Indolocarbazole, Pyrrolopyrimidine, Dianilinopyrimidine.
Poly [ADPribose] polymerase (PARP) inhibitors, illustrated by Benzamide, Phthalazinone, Tricyclic indole, Benzimidazole, Indazole, Pyrrolocarbazole, Phthalazinone, Isoindolinone.

Active agents useable in the present disclosure are not limited to those drug classes or particular agents enumerated above. Different discovery platforms continue to yield new agents that are directed at unique molecular signatures of cancer cells; indeed, thousands of such chemical and biological drugs have been discovered, only some of which are listed here. Yet, the surprising capability of intact, bacterially derived minicells to accommodate packaging of a diverse variety of active agents, hydrophilic or hydrophobic, means that essentially any such drug, when packaged in minicells, has the potential to treat a cancer.

The minicells of the present invention may comprise antineoplastic agent cargo. After the minicell binds a tumor cell and is macropinocytosed by the tumor cell, this cargo may be released into the tumor cell cytoplasm upon degradation of the minicell. Any small molecule, peptide, biologic, super-cytotoxic drug or nucleic acid that can treat the tumor may chosen as the antineoplastic agent.

In one embodiment, the anti-neoplastic agent is selected from the group consisting of a radionuclide, a chemotherapy drug, a functional nucleic acid, and a polynucleotide from which a functional nucleic acid can be transcribed. In one embodiment, the anti-neoplastic agent is a supertoxic chemotherapy drug. In one embodiment, the supertoxic chemotherapy drug is selected from the group consisting of morpholinyl anthracycline, a maytansinoid, ducarmycin, auristatins, calicheamicins (DNA damaging agents), α-amanitin (RNA polymerase II inhibitor), centanamycin, pyrrolobenzodiazepine, streptonigtin, nitrogen mustards, nitrosorueas, alkane sulfonates, pyrimidine analogs, purine analogs, antimetabolites, folate analogs, anthracyclines, taxanes, vinca alkaloids, topoisomerase inhibitors, hormonal agents, and a combination thereof. In one embodiment, the morpholinyl anthracycline is selected from the group consisting of nemorubicin, PNU-159682, idarubicin, daunorubicin; caminomycin, and oxorubicin. In one embodiment, the supertoxic chemotherapy drug is PNU-159682.

In one embodiment, the functional nucleic acid is selected from the group consisting of a siRNA, a miRNA, a shRNA, a lincRNA, an antisense RNA, and a ribozyme. In one embodiment, the functional nucleic acid inhibits a gene that promotes tumor cell proliferation, angiogenesis or resistance to chemotherapy and/or that inhibits apoptosis or cell cycle arrest. In some embodiments, the siRNA inhibits ribonucleotide reductase M1 (RRM1) expression. In some embodiments, the siRNA inhibits Polo like kinase 1 (Plk1) expression. In some embodiments, the miRNA is miRNA16a.

The “small molecule” subcategory of antineoplastic agents encompasses organic compounds characterized by having (i) an effect on a biological process and (ii) a relatively low molecular weight, compared to a macromolecule. Small molecules typically are about 800 Daltons or less, where “about” indicates that the qualified molecular-weight value is subject to variances in measurement precision and to experimental error on the order of several Daltons or tens of Daltons. Thus, a small molecule can have a molecular weight of about 900 Daltons or less, about 800 or less, about 700 or less, about 600 or less, about 500 or less, or about 400 Daltons or less. More specifically, a small molecule can have a molecular weight of about 400 Daltons or more, about 450 Daltons or more, about 500 Daltons or more, about 550 Daltons or more, about 600 Daltons or more, about 650 Daltons or more, about 700 Daltons or more, or about 750 Daltons or more. In another embodiment, the small molecule packaged into the minicells has a molecular weight between about 400 and about 900 Daltons, between about 450 and about 900 Daltons, between about 450 and about 850 Daltons, between about 450 and about 800 Daltons, between about 500 and about 800 Daltons, or between about 550 and about 750 Daltons.

For purposes of this description a “biologic” is defined, to denote any biologically active macromolecule that can be created by a biological process, exclusive of “functional nucleic acids,” discussed herein, and polypeptides that by size qualify as small molecule drugs, as defined above. The “biologic” subcategory of antineoplastic thus is exclusive of and does not overlap with the small molecule drug and functional nucleic acid subcategories. Illustrative of biologics are therapeutic proteins and antibodies, whether natural or recombinant or synthetically made, e.g., using the tools of medicinal chemistry and drug design.

The phrases “highly toxic chemotherapy drug,” “super-cytotoxic drug,” or “supertoxic chemotherapy drug” in this description are used interchangeably and refer to chemotherapy drugs that have a relative low lethal dose as compared to their effective dose for a targeted cancer. Thus, in one aspect a highly toxic chemotherapy drug has a median lethal dose (LD₅₀) that is lower than its median effective dose (ED₅₀) for a targeted cancer such as (1) a cancer type for which the drug is designed, (2) the first cancer type in which a pre-clinical or clinical trial is run for that drug, or (3) the cancer type in which the drug shows the highest efficacy among all tested cancers. For instance, a highly toxic chemotherapy drug can have an LD₅₀ that is lower than about 500%, about 400%, about 300%, about 250%, about 200%, about 150%, about 120%, or about 100% of the ED₅₀ of the drug for a targeted cancer. In another aspect, a highly toxic chemotherapy drug has a maximum sub-lethal dose (i.e., the highest dose that does not cause serious or irreversible toxicity) that is lower than its minimum effective dose for a targeted cancer, e.g., about 500%, about 400%, about 300%, about 250%, about 200%, about 150%, about 120%, about 100%, about 90%, about 80%, about 70%, about 60% or about 50% of the minimum effective dose.

In some embodiments, the anti-neoplastic agent comprises an agent selected from the group consisting of actinomycin-D, alkeran, ara-C, anastrozole, BiCNU, bicalutamide, bleomycin, busulfan, capecitabine, carboplatin, carboplatinum, carmustine, CCNU, chlorambucil, cisplatin, cladribine, CPT-11, cyclophosphamide, cytarabine, cytosine arabinoside, cytoxan, dacarbazine, dactinomycin, daunorubicin, dexrazoxane, docetaxel, doxorubicin, DTIC, epirubicin, ethyleneimine, etoposide, floxuridine, fludarabine, fluorouracil, flutamide, fotemustine, gemcitabine, hexamethylamine, hydroxyurea, idarubicin, ifosfamide, irinotecan, lomustine, mechlorethamine, melphalan, mercaptopurine, methotrexate, mitomycin, mitotane, mitoxantrone, oxaliplatin, paclitaxel, pamidronate, pentostatin, plicamycin, procarbazine, steroids, streptozocin, STI-571, tamoxifen, temozolomide, teniposide, tetrazine, thioguanine, thiotepa, tomudex, topotecan, treosulphan, trimetrexate, vinblastine, vincristine, vindesine, vinorelbine, VP-16, xeloda, asparaginase, AIN-457, bapineuzumab, belimumab, brentuximab, briakinumab, canakinumab, cetuximab, dalotuzumab, denosumab, epratuzumab, estafenatox, farletuzumab, figitumumab, galiximab, gemtuzumab, girentuximab (WX-G250), herceptin, ibritumomab, inotuzumab, ipilimumab, mepolizumab, muromonab-CD3, naptumomab, necitumumab, nimotuzumab, ocrelizumab, ofatumumab, otelixizumab, ozogamicin, pagibaximab, panitumumab, pertuzumab, ramucirumab, reslizumab, rituximab, REGN88, solanezumab, tanezumab, teplizumab, tiuxetan, tositumomab, trastuzumab, tremelimumab, vedolizumab, zalutumumab, zanolimumab, 5FC, accutane hoffmann-la roche, AEE788 novartis, AMG-102, anti neoplaston, AQ4N (Banoxantrone), AVANDIA (Rosiglitazone Maleate), avastin (Bevacizumab) genetech, BCNU, biCNU carmustine, CCI-779, CCNU, CCNU lomustine, celecoxib (Systemic), chloroquine, cilengitide (EMD 121974), CPT-11 (CAMPTOSAR, Irinotecan), dasatinib (BMS-354825, Sprycel), dendritic cell therapy, etoposide (Eposin, Etopophos, Vepesid), GDC-0449, gleevec (imatinib mesylate), gliadel wafer, hydroxychloroquine, IL-13, IMC-3G3, immune therapy, iressa (ZD-1839), lapatinib (GW572016), methotrexate for cancer (Systemic), novocure, OSI-774, PCV, RAD001 novartis (mTOR inhibitor), rapamycin (Rapamune, Sirolimus), RMP-7, RTA 744, simvastatin, sirolimus, sorafenib, SU-101, SU5416 sugen, sulfasalazine (Azulfidine), sutent (Pfizer), TARCEVA (erlotinib HCl), taxol, TEMODAR schering-plough, TGF-B anti-sense, thalomid (thalidomide), topotecan (Systemic), VEGF trap, VEGF-trap, vorinostat (SAHA), XL 765, XL184, XL765, zarnestra (tipifarnib), ZOCOR (simvastatin), cyclophosphamide (Cytoxan), (Alkeran), chlorambucil (Leukeran), thiopeta (Thioplex), busulfan (Myleran), procarbazine (Matulane), dacarbazine (DTIC), altretamine (Hexalen), clorambucil, cisplatin (Platinol), ifosafamide, methotrexate (MTX), 6-thiopurines (Mercaptopurine [6-MP], Thioguanine [6-TG]), mercaptopurine (Purinethol), fludarabine phosphate, (Leustatin), flurouracil (5-FU), cytarabine (ara-C), azacitidine, vinblastine (Velban), vincristine (Oncovin), podophyllotoxins (etoposide {VP-16} and teniposide {VM-26}), camptothecins (topotecan and irinotecan), taxanes such as paclitaxel (Taxol) and docetaxel (Taxotere), (Adriamycin, Rubex, Doxil), dactinomycin (Cosmegen), plicamycin (Mithramycin), mitomycin: (Mutamycin), bleomycin (Blenoxane), estrogen and androgen inhibitors (Tamoxifen), gonadotropin-releasing hormone agonists (Leuprolide and Goserelin (Zoladex)), aromatase inhibitors (Aminoglutethimide and Anastrozole (Arimidex)), amsacrine, asparaginase (El-spar), mitoxantrone (Novantrone), mitotane (Lysodren), retinoic acid derivatives, bone marrow growth factors (sargramostim and filgrastim), amifostine, pemetrexed, decitabine, iniparib, olaparib, veliparib, everolimus, vorinostat, entinostat (SNDX-275), mocetinostat (MGCD0103), panobinostat (LBH589), romidepsin, valproic acid, flavopiridol, olomoucine, roscovitine, kenpaullone, AG-024322 (Pfizer), fascaplysin, ryuvidine, purvalanol A, NU2058, BML-259, SU 9516, PD-0332991, P276-00, geldanamycin, tanespimycin, alvespimycin, radicicol, deguelin, BIIB021, cis-imidazoline, benzodiazepinedione, spiro-oxindoles, isoquinolinone, thiophene, 5-deazaflavin, tryptamine, aminopyridine, diaminopyrimidine, pyridoisoquinoline, pyrrolopyrazole, indolocarbazole, pyrrolopyrimidine, dianilinopyrimidine, benzamide, phthalazinone, tricyclic indole, benzimidazole, indazole, pyrrolocarbazole, isoindolinone, morpholinyl anthracycline, a maytansinoid, ducarmycin, auristatins, calicheamicins (DNA damaging agents), α-amanitin (RNA polymerase II inhibitor), centanamycin, pyrrolobenzodiazepine, streptonigtin, nitrogen mustards, nitrosorueas, alkane sulfonates, pyrimidine analogs, purine analogs, antimetabolites, folate analogs, anthracyclines, taxanes, vinca alkaloids, topoisomerase inhibitors, hormonal agents, and any combination thereof.

“Functional nucleic acid” refers to a nucleic acid molecule that, upon introduction into a host cell, specifically interferes with expression of a protein. With respect to treating a tumor, in accordance with the disclosure, it is preferable that a functional nucleic acid payload delivered to tumor cells via intact, bacterially derived minicells inhibits a gene that promotes tumor cell proliferation, angiogenesis or resistance to chemotherapy and/or that inhibits apoptosis or cell-cycle arrest (i.e., a “tumor-promoting gene”).

Oligonucleotide cancer therapies include single- and double-stranded DNA and RNA oligonucleotides, in many cases chemically modified to optimize delivery, pharmacokinetics, and the ability to inhibit gene expression. Mechanisms of action by which nucleotides treat cancer may include transcription inhibition by homologous recombination, triple-helix formation, and promoter sequence decoys, as well as translation inhibition by RNA decoys, antisense oligodeoxynucleotides, and antisense RNA and DNA enzymes.

In some embodiments, the anti-neoplastic agent comprises a functional nucleic acid or a polynucleotide encoding a functional nucleic acid. In some embodiments, the functional nucleic acid inhibits a gene that promotes tumor cell proliferation, angiogenesis or resistance to chemotherapy and/or that inhibits apoptosis or cell cycle arrest. In some embodiments, the functional nucleic acid is selected from the group consisting of siRNA, miRNA, shRNA, lincRNA, antisense RNA, and ribozyme. In some embodiments, the anti-neoplastic agent comprises a polynucleotide encoding a gene that promotes apoptosis.

It is generally the case that functional nucleic acid molecules used in this disclosure have the capacity to reduce expression of a protein by interacting with a transcript for a protein. This category of minicell payload for the disclosure includes regulatory RNAs, such as siRNA, shRNA, short RNAs (typically less than 400 bases in length), micro-RNAs (miRNAs), ribozymes and decoy RNA, antisense nucleic acids, and LincRNA, inter alia. In this regard, “ribozyme” refers to an RNA molecule having an enzymatic activity that can repeatedly cleave other RNA molecules in a nucleotide base sequence-specific manner. “Antisense oligonucleotide” denotes a nucleic acid molecule that is complementary to a portion of a particular gene transcript, such that the molecule can hybridize to the transcript and block its translation. An antisense oligonucleotide can comprise RNA or DNA. The “LincRNA” or “long intergenic non-coding RNA” rubric encompasses non-protein coding transcripts longer than 200 nucleotides. LincRNAs can regulate the transcription, splicing, and/or translation of genes, as discussed by Khalil et al., Proc Nat'l Acad. USA 106: 11667-72 (2009), for instance.

Each of the types of regulatory RNA can be the source of functional nucleic acid molecule that inhibits a tumor-promoting gene as described above and, hence, that is suitable for use according to the present disclosure. Thus, in one preferred embodiment of the disclosure the intact minicells carry siRNA molecules mediating a post-transcriptional, gene-silencing RNA interference (RNAi) mechanism, which can be exploited to target tumor-promoting genes. For example, see MacDiarmid et al., Nature Biotech. 27: 645-51 (2009) (antibody-presenting minicells deliver, with chemotherapy drug, siRNAs that counter developing resistance to drug), and Oh and Park, Advanced Drug Delivery Rev. 61: 850-62 (2009) (delivery of therapeutic siRNAs to treat breast, ovarian, cervical, liver, lung and prostate cancer, respectively).

As noted, “siRNA” generally refers to double-stranded RNA molecules from about 10 to about 30 nucleotides long that are named for their ability specifically to interfere with protein expression. Preferably, siRNA molecules are 12-28 nucleotides long, more preferably 15-25 nucleotides long, still more preferably 19-23 nucleotides long and most preferably 21-23 nucleotides long. Therefore, siRNA molecules can be 12, 13, 14, 15, 16, 17, 18, 19, 20, 21, 22, 23, 24, 25, 26, 27 28 or 29 nucleotides in length.

The length of one strand designates the length of an siRNA molecule. For instance, an siRNA that is described as 21 ribonucleotides long (a 21-mer) could comprise two opposing strands of RNA that anneal for 19 contiguous base pairings. The two remaining ribonucleotides on each strand would form an “overhang.” When an siRNA contains two strands of different lengths, the longer of the strands designates the length of the siRNA. For instance, a dsRNA containing one strand that is 21 nucleotides long and a second strand that is 20 nucleotides long, constitutes a 21-mer.

Tools to assist the design of siRNA specifically and regulatory RNA generally are readily available. For instance, a computer-based siRNA design tool is available on the internet at www.dharmacon.com.

In another preferred embodiment, the intact minicells of the present disclosure carry miRNAs, which, like siRNA, are capable of mediating a post-transcriptional, gene-silencing RNA interference (RNAi) mechanism. Also like siRNA, the gene-silencing effect mediated by miRNA can be exploited to target tumor-promoting genes. For example, see Kota et al., Cell 137: 1005-17 (2009) (delivery of a miRNA via transfection resulted in inhibition of cancer cell proliferation, tumor-specific apoptosis and dramatic protection from disease progression without toxicity in murine liver cancer model), and Takeshita, et al., Molec. Ther., 18: 181-87 (2010) (delivery of synthetic miRNA via transient transfection inhibited growth of metastatic prostate tumor cells on bone tissues).

Although both mediate RNA interference, miRNA and siRNA have noted differences. In this regard, “miRNA” generally refers to a class of 17- to 27-nucleotide single-stranded RNA molecules (instead of double-stranded as in the case of siRNA). Therefore, miRNA molecules can be 17, 18, 19, 20, 21, 22, 23, 24, 25, 26, 27 nucleotides in length. Preferably, miRNA molecules are 21-25 nucleotide long.

Another difference between miRNAs and siRNAs is that the former generally do not fully complement the mRNA target. On the other hand, siRNA must be completely complementary to the mRNA target. Consequently, siRNA generally results in silencing of a single, specific target, while miRNA is promiscuous.

Additionally, although both are assembled into RISC (RNA-induced silencing complex), siRNA and miRNA differ in their respective initial processing before RISC assembly. These differences are described in detail in Chu et al., PLoS Biology, 4: 1122-36 (2006), and Gregory et al., Methods in Molecular Biology, 342: 33-47 (2006).

A number of databases serve as miRNA depositories. For example, see miRBase (www.mirbase.org) and tarbase (http://diana.cslab.ece.ntua.gr/DianaToolsNew/index.php?r=tarbase/index). In conventional usage, miRNAs typically are named with the prefix “-mir,” combined with a sequential number. For instance, a new miRNA discovered after mouse mir-352 will be named mouse mir-353.

Again, tools to assist the design of regulatory RNA including miRNA are readily available. In this regard, a computer-based miRNA design tool is available on the internet at wmd2.weigelworld.org/cgi-bin/mirnatools.pl.

As noted above, a functional nucleic acid employed in the disclosure can inhibit a gene that promotes tumor cell proliferation, angiogenesis or resistance to chemotherapy. The inhibited gene also can itself inhibit apoptosis or cell cycle arrest. Examples of genes that can be targeted by a functional nucleic acid are provided below.

Functional nucleic acids of the disclosure preferably target the gene or transcript of a protein that promotes drug resistance, inhibits apoptosis or promotes a neoplastic phenotype. Successful application of functional nucleic acid strategies in these contexts have been achieved in the art, but without the benefits of minicell vectors. See, e.g., Sioud (2004), Caplen (2003), Nieth et al. (2003), Caplen and Mousses (2003), Duxbury et al. (2004), Yague et al. (2004), and Duan et al. (2004).

Proteins that contribute to drug resistance constitute preferred targets of functional nucleic acids. The proteins may contribute to acquired drug resistance or intrinsic drug resistance. When diseased cells, such as tumor cells, initially respond to drugs, but become refractory on subsequent treatment cycles, the resistant phenotype is acquired. Useful targets involved in acquired drug resistance include ATP binding cassette transporters such as P-glycoprotein (P-gp, P-170, PGY1, MDR1, ABCB1, MDR-associated protein, Multidrug resistance protein 1), MDR-2 and MDR-3. MRP2 (multi-drug resistance associated protein), BCR-ABL (breakpoint cluster region—Abelson protooncogene), a STI-571 resistance-associated protein, lung resistance-related protein, cyclooxygenase-2, nuclear factor kappa, XRCC1 (X-ray cross-complementing group 1), ERCC1 (Excision cross-complementing gene), GSTP1 (Glutathione S-transferase), mutant 0-tubulin, and growth factors such as IL-6 are additional targets involved in acquired drug resistance.

Particularly useful targets that contribute to drug resistance include ATP binding cassette transporters such as P-glycoprotein, MDR-2, MDR-3, BCRP, APT11a, and LRP.

Useful targets also include proteins that promote apoptosis resistance. These include Bcl-2 (B cell leukemia/lymphoma), Bcl-X_{L}, A1/Bfl 1, focal adhesion kinase, dihydrodiol dehydrogenase, and p53 mutant protein.

Useful targets further include oncogenic and mutant tumor suppressor proteins. Illustrative of these are β-Catenin, PKC-α (protein kinase C), C-RAF, K-Ras (V12), DP97 Dead box RNA helicase, DNMT1 (DNA methyltransferase 1), FLIP (Flice-like inhibitory protein), C-Sfc, 53BPI, Polycomb group protein EZH2 (Enhancer of zeste homologue), ErbB1, HPV-16 E5 and E7 (human papillomavirus early 5 and early 7), Fortilin & MCI1P (Myeloid cell leukemia 1 protein), DIP13α (DDC interacting protein 13a), MBD2 (Methyl CpG binding domain), p21, KLF4 (Kruppel-like factor 4), tpt/TCTP (Translational controlled tumor protein), SPK1 and SPK2 (Sphingosine kinase), P300, PLK1 (Polo-like kinase-1), Trp53, Ras, ErbB1, VEGF (Vascular endothelial growth factor), BAG-1 (BCL2-associated athanogene 1), MRP2, BCR-ABL, STI-571 resistance-associated protein, lung resistance-related protein, cyclooxygenase-2, nuclear factor kappa, XRCC1, ERCC1, GSTP1, mutant (3-tubulin, and growth factors.

Also useful as targets are global regulatory elements exemplified by the cytoplasmic polyadenylation element binding proteins (CEPBs). For instance, CEPB4 is overexpressed in glioblastoma and pancreatic cancers, where the protein activates hundreds of genes associated with tumor growth, and it is not detected in healthy cells (Oritz-Zapater et al., 2011). In accordance with the present description, therefore, treatment of a glioblastoma could be effected via administration of a composition containing intact, bacterially derived minicells that encompass an agent that counters overexpression of CEPB4, such as an siRNA or other functional nucleic acid molecule that disrupts CEPB4 expression by the tumor cells.

The theranostic composition can contain at most about 1 mg of the anti-neoplastic agent. Alternatively, the amount of the anti-neoplastic agent can be at most about 750 μg, 500 μg, 250 μg, 100 μg, 50 μg, 10 μg, 5 μg, 1 μg, 0.5 μg, or 0.1 μg. In another aspect, the theranostic composition contains an anti-neoplastic agent having an amount of less than about 1/1,000, or alternatively less than about 1/2,000, 1/5,000, 1/10,000, 1/20,000, 1/50,000, 1/100,000, 1/200,000 or 1/500,000 of the therapeutically effective amount of the drug when used without being packaged to into minicells. Pursuant to yet another aspect of the disclosure, the theranostic composition can contain at least about 1 nmol of the chemotherapeutic drug. Accordingly, the disclosure also encompasses embodiments where the amount of the anti-neoplastic agent is at least about 2 nmol, about 3 nmol, about 4 nmol, about 5 nmol, about 10 nmol, about 20 nmol, about 50 nmol, about 100 nmol, and about 800 nmol, respectively.

In some embodiments, the minicells comprise from about 5×10³ to about 5×10⁴ molecules of the super-cytotoxic drug. In some embodiments, the minicells comprise from about 5×10⁴ to about 5×10⁵ molecules of the super-cytotoxic drug. In some embodiments, the minicells comprise from about 5×10⁵ to about 1.5×10⁶ molecules of the super-cytotoxic drug. In some embodiments, the minicells comprise from about 1.5×10⁶ to about 5×10⁷ molecules of the super-cytotoxic drug. In some embodiments, the minicells comprise from about 5×10⁷ to about 5×10⁸ molecules of the super-cytotoxic drug. In some embodiments, the minicells comprise from about 5×10⁸ to about 5×10⁹ molecules of the super-cytotoxic drug. In some embodiments, the minicells comprise from about 5×10⁹ to about 5×10¹⁰ molecules of the super-cytotoxic drug.

Whether antineoplastic agent is a small molecule, functional nucleic acid, or a biologic, moreover, certain molecules used outside the context of the minicells disclosed herein, that are designed for chemotherapeutic purposes, nevertheless fail during pre-clinical or clinical trials due to unacceptable toxicity or other safety concerns. The present directed to packaging antineoplastic in a minicell, followed by systemic delivery to a tumor patient, results in delivery of the antineoplastic to tumor cells. Further, even after the tumor cells are broken up and the antineoplastic-containing cytoplasm is released to the nearby normal tissue, the result is not toxicity to normal tissue. This is because the antineoplastic is already bound to the tumor cellular structures, such as DNA, and can no longer attack normal cells. Accordingly, the present invention is particularly useful for delivery of highly toxic chemotherapy drugs to a tumor patient.

### II. Advantages of Using Minicells Including the Mono- and Bispecific Ligands

Despite their specific targeting ability, the ability to carry radio-imaging agents, and desirable pharmacokinetics, native peptides are seldom directly used for in vivo imaging since peptides have several limitations. First, peptides usually have a very short in vivo biological half-life of around several minutes. In addition, peptides suffer from enzymatic degradation as well as fast renal clearance and hence lose their bioactivity even before reaching the intended target.

The present invention allows the radiolabel to be attached to a monospecific ligand (polypeptide) which then binds the minicell second surface component. This minicell theranostic does not have the limitations that small peptides have since the minicell is about 400 nm in diameter and therefore not subject to rapid clearance from general blood circulation. In some embodiments, the minicell has a diameter of about 100 nm to about 600 nm. The theranostic can rapidly enter into the tumor microenvironment and hence is able to specifically image the tumor via the radiolabel attached to the minicell. Simultaneously, the radiation emitted by the radiolabel specifically irradiates the tumor cells to achieve anti-tumor therapeutic efficacy.

The present invention comprises a monospecific ligand which binds to a second surface component on the minicell and a bispecific ligand which binds to a first surface component on the minicell. In some embodiments, the first surface component and/or the second surface component of the minicell may comprise carbohydrates, peptides, or a combination thereof. In some embodiments, the first surface component and/or the second surface component comprises lipopolysaccharide (LPS). The O-polysaccharide component of the lipopolysaccharide may serve as the moiety to which the monospecific (or bispecific) ligand binds in order to fasten the ligand to the minicell. Embodiments of the invention have a first surface component (to where the bispecific ligand binds) and a second surface component (to where the monospecific ligand binds). In some embodiments, the first surface component and the second surface component are different surface components, e.g. at two different sites of the minicell and having substantially different structure or sequences.

In some embodiments, the first surface component comprises a first polypeptide and the second surface component comprises a second polypeptide, wherein the first polypeptide and the second polypeptide share greater than about 50, about 60, about 70, about 80, about 90, about 95, or about 99% sequence identity with each other. In some embodiments, the first surface component comprises a first polynucleotide and the second surface component comprises a second polynucleotide, wherein the first polynucleotide and the second polynucleotide share greater than about 50, about 60, about 70, about 80, about 90, about 95, or about 99% sequence identity with each other.

In some embodiments, the first surface component comprises a first polypeptide and the second surface component comprises a second polypeptide, wherein the first polypeptide and the second polypeptide share less than about 50, about 60, about 70, about 80, about 90, about 95, or about 99% sequence identity with each other. In some embodiments, the first surface component comprises a first polynucleotide and the second surface component comprises a second polynucleotide, wherein the first polynucleotide and the second polynucleotide share less than about 50, about 60, about 70, about 80, about 90, about 95, or about 99% sequence identity with each other.

The minicells also comprise a bispecific ligand which binds a tumor cell surface receptor. In some embodiments, the bispecific ligand comprises (i) a first arm with binding specificity for the first minicell surface component; and (ii) a second arm with binding specificity for a tumor cell surface receptor. This allows for targeted delivery to the tumor cell of minicell antineoplastic agent cargo and radiation from the imaging agent.

In some embodiments, the bispecific ligand comprises a polypeptide, aptamer, carbohydrate, or combination thereof. In some embodiments, the bispecific ligand has a specificity to a non-phagocytic mammalian tumor cell surface receptor. In some embodiments, the non-phagocytic mammalian tumor cell surface receptor comprises a tumor cell antigen. In some embodiments, the bispecific ligand comprises an antibody that specifically recognizes the tumor cell antigen.

### III. Methodology

A. Attaching Radiolabels to Minicells

There are several ways to attach radiolabels to theranostic minicells. These methods include but are not limited to, (1) conjugating the radiolabel directly to the monospecific ligand and attaching the radiolabeled ligand to a surface component of the minicell; or (2) packaging the radiolabel in synthetic nanoparticles and then conjugating the nanoparticles to the minicell surface monospecific ligand.

In some embodiments, the radio-imaging agent is comprised within a synthetic nanoparticle, and the synthetic nanoparticle can be conjugated to the monospecific ligand.

Several pre-existing radiolabeled particles can be attached to the monospecific antibody attached to the surface of the minicell. Examples include but are not limited to the following examples [Barros et al., 2012].

Liposomes: liposomes such as ^{99m}Tc-PEG liposomes, (¹¹¹In)-loaded liposomes, ¹¹¹In-DTPA-labeled PEGylated liposomes, ¹⁸⁸Re-N N-bis(2-mercaptoethyl)-N′,N′-diethylethylenediamine (BMEDA)-labeled PEGylated liposomes, and ⁶⁴Cu-loaded PEGylated liposomes may be used.

Iron oxide nanoparticles: iron oxide nanoparticles may be used including those with a magnetic particle core (typically magnetite, Fe₃O₄, or maghemite, Fe₂O₃) coated with a hydrophilic and biocompatible polymer such as PEG, dextran, alginate, and poly(d l-lactide-co-glycolide); a porous biocompatible polymer in which iron oxide nanoparticles are entrapped within the polymer matrix; radiolabeled iron oxide nanoparticle conjugated with cyclic arginine-glycine-aspartic (RGD) that is functionalized with DOTA for labeling with ⁶⁴Cu; and dextran-coated and DTPA-modified magnetofluorescent 20-nm nanoparticle radiolabeled with ⁶⁴Cu.

Gold nanoparticles: gold nanoparticles may also be used such as gold nanoparticles coated with PEG2k-DOTA for ⁶⁴Cu chelation; ^{99m}Tc-labeled gold nanoparticles conjugated with c[RGDfk(C)] for tumor imaging; and ¹²⁵I-labeled gold nanorods.

Micelles: micelles constitute another class of nanoparticles that may carry a radiolabel, examples include micelles labeled with ¹¹¹In and a near-infrared fluorescent indocyanine (Cy7)-like dye; multifunctional micelles with a hyperbranched amphiphilic block copolymer conjugated with cRGD peptide (for integrin αᵥβ₃ target) or NOTA (a macrocyclic chelator for ⁶⁴Cu-labeling and PET imaging).

Carbon based nanoparticles: carbon based nanoparticles may also be used to carry the radiolabel. These include carbon nanotubes (single- and multiwalled), fullerenes, perfluorocarbon nanoemulsions, and graphene oxide nanoparticles.

Radiolabeled Nanoparticles: In one embodiment, the invention provides a previously described nanoparticle (US 2008/0095699 A1) comprising: (a) a core comprising a magnetic material and having a surface; (b) a poly (beta-amino ester) coupled to the surface of the core, wherein the poly (beta-amino ester) comprises a poly (beta-amino ester) backbone having one or more therapeutic agents and one or more anchoring groups covalently coupled thereto; and (c) a diagnostic agent.

In other embodiments, the above nanoparticles further comprise a polyalkylene oxide group (e.g., polyethylene oxide) pendant from the poly (beta-amino ester).

The imaging dye or radiolabel can be packaged in the nanoparticle where the particle is less than about 10 nm in size. A monospecific ligand is conjugated to the radiolabel-packaged nanoparticle where a moiety of the monospecific ligand has specificity for the minicell surface carbohydrate or protein. Thus, the minicell including as monospecific antibody is coated with the nanoparticles packaged with a radio-imaging agent.

At present, the magnetic iron oxide nanoparticle (IONP) or superparamagnetic iron oxide nanoparticle (SPION) is one of the few nanoparticle platforms that are being developed as theranostic agents. [Huang et al., 2016; Laurent et al., 2008; 2009].

Significant discoveries have been made in the production of IONPs with different core sizes, surface functions, MRI contrast properties and drug-loading capacity [Boni et al., 2008; Laurent et al., 2008].

A marked feature of magnetic IONPs is the ability of the production of controlled and uniform core size nanoparticles with a size-dependent magnetization. Ultrafine IONPs at a sub-5-nm core size have been developed for improved delivery efficiency and dual MRI contrast [Huang et al., 2014; 2016].

IONP sizes developed by different groups are all within a size range (less than 100 nm; Sun and Zeng, 2002; Xie et al., 2008; Huang et al., 2014) to be useful for conjugation to theranostic minicells. Ultrasmall IONPs with core size (<10 nm) and ultrafine IONPs (<5 nm) have been synthesized and characterized [Huang et al., 2014; Wang et al., 2014] and these are preferred size for use with theranostic minicells.

Various targeting ligands have been conjugated to magnetic IONPs. Typically, targeting ligands include antibodies or engineered antibody fragments, natural ligands, peptides, structured DNA and RNA molecules, and small molecules [Markman et al., 2013]. These targeting ligands can be engineered to have specificity for a minicell surface component (the second surface component) and serve as the monospecific ligand.

The nanoparticle includes a core material. For magnetic resonance imaging applications, the core material is a material having magnetic resonance imaging activity (e.g., the material is paramagnetic). In certain embodiments, the core material is a magnetic material. In other embodiments, the core material is a semiconductor material. Representative core materials include ferrous oxide, ferric oxide, silicon oxide, polycrystalline silicon oxide, silicon nitride, aluminum oxide, germanium oxide, Zinc selenide, tin dioxide, titanium, titanium dioxide, nickel titanium, indium tin oxide, gadolinium oxide, stainless steel, gold, and mixtures thereof.

Suitable nanoparticles have a physical size of less than about 30 nm. In certain embodiments, the nanoparticles have a physical size from about 10 to about 30 nm. In other embodiments, the nanoparticles have a physical size from about 10 to about 20 nm. As used herein, the term “physical size’ refers the overall diameter of the nanoparticle, including core (as determined by TEM) and coating thickness. Suitable particles have a mean core size of from about 2 to about 25 nm. In certain embodiments, the nanoparticles have a mean core size of about 7 nm. As used herein, the term “mean core size” refers to the core size determined by TEM.

Fluorophore labeling of the monospecific ligand: Suitable diagnostic imaging agents include optical agents, such as fluorescent agents that emit light in the visible and near infrared (e.g., fluorescein and cyanine derivatives). Suitable fluorescent agents include fluorescein and derivatives, rhodamine and derivatives, and cyanines. Representative fluorescent agents include fluorescein, OREGON GREEN 488, ALEXA FLUOR 555, ALEXA FLUOR 647, ALEXA FLUOR 680, Cy5, Cy5.5, and Cy7.

The design of fluorophore labeled peptides is similar to radiolabeled peptides except that fluorophores are used to replace radionuclides. Various dyes are commercially available (for example, Cyanine dyes from GE Healthcare and Alexa Fluor dyes from Invitrogen). Cy5.5 has been conjugated to RGD monomer, dimer and tetramer for in vivo optical imaging [Cheng et al., 2005].

Although the fluorescence imaging has the advantages of high resolution, non-invasive and safe detection, the use of fluorophores in vivo is limited by the light penetration and tissue autofluorescence. The introduction of hybrid derivatives containing both a fluorescent tag and a radioactive label may be a way to overcome the limitation. Zhu et al. have conjugated cyclic RGD peptide with both DOTA for ⁶⁴Cu labeling and a near-infrared ZW-1 dye [Zu et al., 2012]. The tumor region in preclinical xenograft models can be clearly seen via both optical imaging and PET imaging with high tumor-to-background contrast.

B. Methods of Increasing In Vivo Circulation Time for the Theranostic

PEGylation of Minicell Theranostic: The complete minicell structure described above can further be PEGylated to reduce uptake by professional phagocytic cells when the minicells are in circulation in the blood stream of a patient. This would increase circulation time and hence more minicells would extravasate into the tumor microenvironment via the tumor-associated leaky vasculature. This would increase the target:background ratio.

Additionally, an anti-biofouling polymer-PEO-blockpoly(γ-methacryloxypropyltrimethoxysilane) (PEOb-PγMPS) has been developed to coat IONPs [Chen et al., 2010]. In comparison with other surface modifications, PEO-b-PγMPS-coated nanoparticles had significantly reduced nonspecific binding to serum proteins and uptake by macrophages in the liver and spleen. In a recent study, HER2 antibody and single-chain anti-EGFR antibody-conjugated PEO-b-PγMPS-IONPs showed a high level of the accumulation in tumors following systemic delivery, suggesting the potential for using this system to improve efficiency of tumor-targeted delivery [Chen et al., 2013].

The minicells of the present invention may be coated with such polymers. Thus, in some embodiments, the minicell comprises a polymer film or coat. In some embodiments, the polymer is pharmaceutically acceptable. In some embodiments, the polymer film or coat is opsonization-reducing. In some embodiments, the polymer film or coat reduces or minimizes macrophage uptake of the composition.

In some embodiments, the polymer film or coat comprises a polymer selected from the group consisting of a polyethylene glycol, polymer-PEO-blockpoly(γ-methacryloxypropyltrimethoxysilane) (PEOb-PγMPS), and (trimethoxysilyl)propyl methacrylate-PEG-methacrylate.

New surface modifications for IONPs have been shown to reduce nonspecific interactions with serum proteins and macrophage uptake. For instance, coating SPIONs with an anti-biofouling copolymeric system, named (trimethoxysilyl)propyl methacrylate-PEG-methacrylate increased biostability of SPIONs and reduced ‘opsonization’ process [Lee et al., 2006; Park et al., 2007]. Resulting poly(trimethoxysilyl)propyl methacrylate-PEG-methacrylate-coated SPION had excellent biocompatibility, tumor-targeting ability and long-circulated time in vivo.

C. Packaging of Anti-Neoplastic Agent into Minicells

Anti-neoplastic agents, such as proteins and functional nucleic acids, that can be encoded by a nucleic acid, can be introduced into minicells by transforming into the parental bacterial cell a vector, such as a plasmid, that encodes the anti-neoplastic agent. When a minicell is formed from the parental bacterial cell, the minicell retains certain copies of the plasmid and/or the expression product, the anti-neoplastic agent. More details of packaging an expression product into a minicell is provided in WO 03/033519, the content of which is incorporated into the present disclosure in its entirety by reference.

Data presented in WO 03/033519 demonstrated, for example, that recombinant minicells carrying mammalian gene expression plasmids can be delivered to phagocytic cells and to non-phagocytic cells. The application also described the genetic transformation of minicell-producing parent bacterial strains with heterologous nucleic acids carried on episomally-replicating plasmid DNAs. Upon separation of parent bacteria and minicells, some of the episomal DNA segregated into the minicells. The resulting recombinant minicells were readily engulfed by mammalian phagocytic cells and became degraded within intracellular phagolysosomes. Moreover, some of the recombinant DNA escaped the phagolysosomal membrane and was transported to the mammalian cell nucleus, where the recombinant genes were expressed.

Nucleic acids also can be packaged into minicells directly. Thus, a nucleic acid can be packaged directly into intact minicells by co-incubating a plurality of intact minicells with the nucleic acid in a buffer. The buffer composition can be varied, as a function of conditions well known in this field, in order to optimize the loading of the nucleic acid in the intact minicells. The buffer also may be varied in dependence on the nucleotide sequence and the length of the nucleic acid to be loaded in the minicells. Once packaged, the nucleic acid remains inside the minicell and is protected from degradation. Prolonged incubation studies with siRNA-packaged minicells incubated in sterile saline showed, for example, no leakage of siRNAs.

In other embodiments, multiple nucleic acids directed to different mRNA targets can be packaged in the same minicell. Such an approach can be used to combat drug resistance and apoptosis resistance. For example, cancer patients routinely exhibit resistance to chemotherapeutic drugs. Such resistance can be mediated by over-expression of genes such as multi-drug resistance (MDR) pumps and anti-apoptotic genes, among others. To combat this resistance, minicells can be packaged with therapeutically significant concentrations of functional nucleic acid to MDR-associated genes and administered to a patient before chemotherapy. Furthermore, packaging into the same minicell multiple functional nucleic acid directed to different mRNA targets can enhance therapeutic success since most molecular targets are subject to mutations and have multiple alleles. More details of directly packaging a nucleic acid into a minicell is provided in WO 2009/027830, the contents of which are incorporated into the present disclosure in its entirety by reference.

Small molecules, whether hydrophilic or hydrophobic, can be packaged in minicells by creating a concentration gradient of the small molecule between an extracellular medium containing minicells and the minicell cytoplasm. When the extracellular medium contains a higher small molecule concentration than the minicell cytoplasm, the small molecule naturally moves down this concentration gradient, into the minicell cytoplasm. When the concentration gradient is reversed, however, the small molecule does not move out of the minicells.

To load minicells with small molecules that normally are not water soluble, the small molecule initially can be dissolved in an appropriate solvent. For example, Paclitaxel can be dissolved in a 1:1 blend of ethanol and cremophore EL (polyethoxylated castor oil), followed by a dilution in PBS to achieve a solution of Paclitaxel that is partly diluted in aqueous media and carries minimal amounts of the organic solvent to ensure that the small molecule remains in solution. Minicells can be incubated in this final medium for small molecule loading. Thus, the inventors discovered that even hydrophobic small molecules can diffuse into the cytoplasm or the membrane of minicells to achieve a high and therapeutically significant cytoplasmic small molecule load. This is unexpected because the minicell membrane is composed of a hydrophobic phospholipid bilayer, which would be expected to prevent diffusion of hydrophobic molecules into the cytoplasm.

Example 10 of U.S. patent application Ser. No. 15/790,885, the entire disclosure of which is hereby incorporated by reference, demonstrates the loading into minicells of a diversity of representative small molecules, illustrating different sizes and chemical properties: Doxorubicin, Paclitaxel, Fluoro-paclitaxel, Cisplatin, Vinblastine, Monsatrol, Thymidylate synthase (TS) inhibitor OSI-7904, Irinotecan, 5-Fluorouracil, Gemcitabine, and Carboplatin. The resultant, small molecule-packaged minicells show significant anti-tumor efficacy, in vitro and in vivo. This clearly demonstrates the effectiveness and versatility of the minicell loading methods.

D. Methods of Imaging and Treating Tumors

The theranostic compositions of the present invention are useful in the treatment and imaging of tumors. Targeted delivery of imaging agent facilitates imaging of tumors using methods including SPECT, MRI, and PET discussed above. The proximal delivery of radiation by minicells bound to tumors, as well as the endocytosis of minicells by the tumor cell and subsequent delivery of antineoplastic and radionuclide to the cytoplasm of the cancer cell, represents a strategy by which tumor cells may be simultaneously imaged and treated.

For treating a tumor, a theranostic composition of the disclosure would be delivered in a dose or in multiple doses that in toto afford a level of in-tumor irradiation that is sufficient at least to reduce tumor mass, if not eliminate the tumor altogether. The progress of treatment can be monitored along this line, on a case-by-case basis. In general terms, however, the amount of radioactivity packaged in the composition typically will be on the order of about 30 to 50 Gy, although the invention also contemplates a higher amount of radioactivity, say, about 50 to 100 Gy, which gives an overall range between about 30 Gy and about 100 Gy. In another embodiment, the theranostic composition contains from about 20 to 40 Gy, or about 10 to 30 Gy, or about 1 to about 20 Gy, or less than 10 Gy.

In one embodiment, a method of imaging a tumor in a subject is provided, the method comprising administering systemically to the subject the theranostic composition of any embodiment disclosed herein, wherein the theranostic composition comprises a diagnostically effective amount of the radio-imaging agent. In general, the total effective imaging radiation dose depends upon the part of the body where the tumor is located, and ranges from about 0.4 to about 262 mSv (millisievert).

In one embodiment, a method for treating a tumor in a subject is provided, the method comprising administering systemically to the subject the theranostic composition of any embodiment disclosed herein, wherein the composition comprises a therapeutically effective amount of the radio-imaging agent and a therapeutically effective amount of the anti-neoplastic agent. In general, the amount of the radioimaging agent ranges from about 0.4 mSv to about 262 mSv.

In one embodiment, a method of imaging and treating a tumor in a subject is provided, the method comprising administering systemically to the subject the theranostic composition of any embodiment of the invention disclosed herein, wherein the composition comprises: (a) a diagnostically effective amount of the radio-imaging agent, wherein the amount of the radio-imaging agent is also therapeutically effective; and (b) a therapeutically effective amount of the anti-neoplastic agent.

In some embodiments, a therapeutically effective amount of the antineoplastic agent comprises from about 5×10³ to about 5×10⁴ molecules of the antineoplastic agent. In some embodiments, a therapeutically effective amount of the antineoplastic agent comprises from about 5×10⁴ to about 5×10⁵ molecules of the antineoplastic agent. In some embodiments, a therapeutically effective amount of the antineoplastic agent comprises from about 5×10⁵ to about 1.5×10⁶ molecules of the antineoplastic agent. In some embodiments, a therapeutically effective amount of the antineoplastic agent comprises from about 1.5×10⁶ to about 5×10⁷ molecules of the antineoplastic agent. In some embodiments, a therapeutically effective amount of the antineoplastic agent comprises from about 5×10⁷ to about 5×10⁸ molecules of the antineoplastic agent. In some embodiments, a therapeutically effective amount of the antineoplastic agent comprises from about 5×10⁸ to about 5×10⁹ molecules of the antineoplastic agent. In some embodiments, a therapeutically effective amount of the antineoplastic agent comprises from about 5×10⁹ to about 5×10¹⁰ molecules of the antineoplastic agent.

In some embodiments, treating a tumor comprises reducing the mass of the tumor. In some embodiments the mass is reduced by about 1 to about 10%. In some embodiments the mass is reduced by about 10 to about 20%. In some embodiments the mass is reduced by about 20 to about 30%. In some embodiments the mass is reduced by about 30 to about 40%. In some embodiments the mass is reduced by about 40 to about 50%. In some embodiments the mass is reduced by about 50 to about 60%. In some embodiments the mass is reduced by about 60 to about 70%. In some embodiments the mass is reduced by about 70 to about 80%. In some embodiments the mass is reduced by about 80 to about 90%. In some embodiments the mass is reduced by about 90 to about 99%. In some embodiments, treating a tumor comprises eradicating the tumor.

In another embodiment, a method of adjusting the signal intensity of an imaged tumor in a subject is provided, the method comprising: (a) systemically administering a first dose of a theranostic composition according to any embodiment herein followed by imaging the tumor; (b) systemically administering a second dose of a theranostic composition according to any embodiment herein followed by imaging the tumor, wherein: (i) the second dose of a theranostic composition comprises a greater amount of the radio-imaging agent per minicell as compared to the first dose; or (ii) the second dose of a theranostic composition comprises a lesser amount of the radio-imaging agent per minicell as compared to the first dose; and (c) comparing the imaging results following (a) and (b) to obtain the adjusted signal intensity. Methods of quantitating image signal intensity are known in the art.

In some embodiments, adjusting the signal intensity comprises increasing the signal intensity by about 1 to about 25%. In some embodiments, adjusting the signal intensity comprises increasing the signal intensity by about 25 to about 50%. In some embodiments, adjusting the signal intensity comprises increasing the signal intensity by about 50 to about 75%. In some embodiments, adjusting the signal intensity comprises increasing the signal intensity by about 75 to about 100%. In some embodiments, adjusting the signal intensity comprises increasing the signal intensity by greater than 100%.

In some embodiments, a greater amount of the radio-imaging agent per minicell as compared to the first dose comprises about 10, 20, 30, 40, 50, 60, 70, 80, 90, 100, 125, 150, 175, or 200% greater. In some embodiments, a lesser amount of the radio-imaging agent per minicell as compared to the first dose comprises about 10, 20, 30, 40, 50, 60, 70, 80, 90, or 99% lesser.

A tumor to be treated can be present in any tissue or organ. Exemplary tumors include but are not limited to brain, stomach, prostate, pituitary, pancreas, lung, liver, spleen, colon, breast, connective tissue (e.g., cartilage, bones, fat, and nerves), ovarian, testicular, blastomas (medulloblastoma, glioblastoma, retinoblastoma, osteoblastoma, and neuroblastoma).

In some embodiments, the tumor does not comprise a brain tumor. In another embodiment, the tumor does not comprise a glioblastoma, astrocytic tumor, oligodendroglial tumor, ependymoma, craniopharyngioma, pituitary tumor, primary lymphoma of the brain, pineal gland tumor, primary germ cell tumor of the brain, or combination thereof. In another embodiment the tumor does not comprise a spleen tumor or a liver tumor.

The subject to which the theranostic composition is administered may be selected from simian, bovine, porcine, murine, rat, avian, reptilian and mammal. In one embodiment, the subject is a human. In another embodiment, the subject is a canine or feline.

Another embodiment, provides use of the theranostic composition of any embodiment herein, for the manufacture of a medicament for treating a tumor. Another embodiment, provides use of the theranostic composition of any embodiment herein, for the manufacture of a medicament for imaging a tumor. Another embodiment, provides use of the theranostic composition of any embodiment herein, for the manufacture of a medicament for imaging and treating a tumor.

E. Formulations and Administration Routes and Schedules

Formulations of a theranostic composition of the disclosure can be presented in unit dosage form, e.g., in ampules or vials, or in multi-dose containers, with or without an added preservative. The formulation can be a solution, a suspension, or an emulsion in oily or aqueous vehicles, and can contain formulatory agents, such as suspending, stabilizing and/or dispersing agents. A suitable solution is isotonic with the blood of the recipient and is illustrated by saline, Ringer's solution, and dextrose solution. Alternatively, formulations can be in lyophilized powder form, for reconstitution with a suitable vehicle, e.g., sterile, pyrogen-free water or physiological saline. The formulations also can be in the form of a depot preparation. Such long-acting formulations can be administered by implantation (for instance, subcutaneously or intramuscularly) or by intramuscular injection. In some embodiments, administering comprises enteral or parenteral administration. In some embodiments administering comprises administration selected from oral, buccal, sublingual, intranasal, rectal, vaginal, intravenous, intramuscular, and subcutaneous injection.

In some aspects, a minicell-containing theranostic composition that includes a therapeutically effective amount of an anti-neoplastic agent is provided. A “therapeutically effective” amount of an anti-neoplastic agent is a dosage of the agent in question, e.g., a siRNA or a super-cytotoxic drug that invokes a pharmacological response when administered to a subject, in accordance with the present disclosure.

In the context of the present disclosure, therefore, a therapeutically effective amount can be gauged by reference to the prevention or amelioration of the tumor or a symptom of tumor, either in an animal model or in a human subject, when minicells carrying a therapeutic payload are administered, as further described below. An amount that proves “therapeutically effective amount” in a given instance, for a particular subject, may not be effective for 100% of subjects similarly treated for the tumor, even though such dosage is deemed a “therapeutically effective amount” by skilled practitioners. The appropriate dosage in this regard also will vary as a function, for example, of the type, stage, and severity of the tumor.

When “therapeutically effective” is used to refer to the number of minicells in a pharmaceutical composition, the number can be ascertained based on what anti-neoplastic agent is packaged into the minicells and the efficacy of that agent in treating a tumor. The therapeutic effect, in this regard, can be measured with a clinical or pathological parameter such as tumor mass. A reduction or reduced increase of tumor mass, accordingly, can be used to measure therapeutic effects.

Formulations within the disclosure can be administered via various routes and to various sites in a mammalian body, to achieve the therapeutic effect(s) desired, either locally or systemically. In a particular aspect, the route of administration is intravenous injection.

In general, formulations of the disclosure can be used at appropriate dosages defined by routine testing, to obtain optimal physiological effect, while minimizing any potential toxicity. The dosage regimen can be selected in accordance with a variety of factors including age, weight, sex, medical condition of the patient; the severity or stage of tumor, the route of administration, and the renal and hepatic function of the patient.

Optimal precision in achieving concentrations of minicell and therapeutic agent within the range that yields maximum efficacy with minimal side effects can and typically will require a regimen based on the kinetics of agent availability to target sites and target cells. Distribution, equilibrium, and elimination of minicells or agent can be considered when determining the optimal concentration for a treatment regimen. The dosage of minicells and therapeutic agent, respectively, can be adjusted to achieve desired effects.

Moreover, the dosage administration of the formulations can be optimized using a pharmacokinetic/pharmacodynamic modeling system. Thus, one or more dosage regimens can be chosen and a pharmacokinetic/pharmacodynamic model can be used to determine the pharmacokinetic/pharmacodynamic profile of one or more dosage regimens. Based on a particular such profile, one of the dosage regimens for administration then can be selected that achieves the desired pharmacokinetic/pharmacodynamic response. For example, see WO 00/67776.

Specifically, the formulations may be administered at least once a week over the course of several weeks. In one embodiment, the formulations are administered at least once a week over several weeks to several months.

More specifically, the formulations may be administered at least once a day for about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or about 31 days. Alternatively, the formulations may be administered about once every day, about once every about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30 or about 31 days or more.

The formulations may alternatively be administered about once every week, about once every about 2, about 3, about 4, about 5, about 6, about 7, about 8, about 9, about 10, about 11, about 12, about 13, about 14, about 15, about 16, about 17, about 18, about 19 or about 20 weeks or more. Alternatively, the formulations may be administered at least once a week for about 2, about 3, about 4, about 5, about 6, about 7, about 8, about 9, about 10, about 11, about 12, about 13, about 14, about 15, about 16, about 17, about 18, about 19 or about 20 weeks or more.

The formulations may alternatively be administered about twice every week, about twice every about 2, about 3, about 4, about 5, about 6, about 7, about 8, about 9, about 10, about 11, about 12, about 13, about 14, about 15, about 16, about 17, about 18, about 19 or about 20 weeks or more. Alternatively, the formulations may be administered at least once a week for about 2, about 3, about 4, about 5, about 6, about 7, about 8, about 9, about 10, about 11, about 12, about 13, about 14, about 15, about 16, about 17, about 18, about 19 or about 20 weeks or more.

Alternatively, the formulations may be administered about once every month, about once every about 2, about 3, about 4, about 5, about 6, about 7, about 8, about 9, about 10, about 11 or about 12 months or more.

The formulations may be administered in a single daily dose, or the total daily dosage may be administered in divided doses of two, three, or four times daily.

In a method in which minicells are administered before a drug, administration of the drug may occur anytime from several minutes to several hours after administration of the minicells. The drug may alternatively be administered anytime from several hours to several days, possibly several weeks up to several months after the minicells.

More specifically, the minicells may be administered at least about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23 or about 24 hours before the drug. Moreover, the minicells may be administered at least about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30 or about 31 days before the administration of the drug. In yet another embodiment, the minicells may be administered at least about 1, about 2, about 3, about 4, about 5, about 6, about 7, about 8, about 9, about 10, about 11, about 12, about 13, about 14, about 15, about 16, about 17, about 18, about 19 or about 20 weeks or more before the drug. In a further embodiment, the minicells may be administered at least about 1, about 2, about 3, about 4, about 5, about 6, about 7, about 8, about 9, about 10, about 11 or about 12 months before the drug.

In another embodiment, the minicell is administered after the drug. The administration of the minicell may occur anytime from several minutes to several hours after administration of the drug. The minicell may alternatively be administered anytime from several hours to several days, possibly several weeks up to several months after the drug.

### IV. Definitions

Unless defined otherwise, all technical and scientific terms used in this description have the same meaning as commonly understood by those skilled in the relevant art.

For convenience, the meaning of certain terms and phrases employed in the specification, examples, and appended claims are provided below. Other terms and phrases are defined throughout the specification.

The singular forms “a,” “an,” and “the” include plural reference unless the context clearly dictates otherwise.

“Cancer,” “neoplasm,” “tumor,” “malignancy” and “carcinoma,” used interchangeably herein, refer to cells or tissues that exhibit an aberrant growth phenotype characterized by a significant loss of control of cell proliferation. The methods and compositions of this disclosure particularly apply to malignant, pre-metastatic, metastatic, and non-metastatic cells.

“Drug” refers to any physiologically or pharmacologically active substance that produces a local or systemic effect in animals, particularly mammals and humans.

The terms “radionuclide,” “radioimaging agent,” “radiolabel,” and the like refer to an atom with an unstable nucleus, i.e., one characterized by excess energy available to be imparted either to a newly created radiation particle within the nucleus or to an atomic electron. Therefore, a radionuclide undergoes radioactive decay, and emits gamma ray(s) and/or subatomic particles. Numerous radionuclides are known in the art and discussed herein. Radionuclides may also be used as an antineoplastic agent.

“Individual,” “subject,” “host,” and “patient,” terms used interchangeably in this description, refer to any mammalian subject for whom diagnosis, treatment, or therapy is desired. The individual, subject, host, or patient can be a human or a non-human animal. Thus, suitable subjects can include but are not limited to non-human primates, cattle, horses, dogs, cats, guinea pigs, rabbits, rats, and mice.

The terms “treatment,” “treating,” “treat,” and the like refer to obtaining a desired pharmacological and/or physiologic effect in a tumor patient. The effect can be prophylactic in terms of completely or partially preventing tumor or symptom thereof and/or can be therapeutic in terms of a partial or complete stabilization or cure for tumor and/or adverse effect attributable to the tumor. Treatment covers any treatment of a tumor in a mammal, particularly a human. A desired effect, in particular, is tumor response, which can be measured as reduction of tumor mass or inhibition of tumor mass increase. In addition to tumor response, an increase of overall survival, progress-free survival, or time to tumor recurrence or a reduction of adverse effect also can be used clinically as a desired treatment effect.

The term “image,” “imaging,” and the like, refer to the visualization of a tumor in a subject. The tumor locale, size, and/or constitution of the tumor may be imaged via imaging using a variety of imaging agents disclosed herein, not limited to, radioimaging agents, dyes, and magnetic imaging agents. Visualization of imaged tumors may be accomplished using any technique known to the skilled artisan, including but not limited to radiography, magnetic resonance imagine (MRI), nuclear medicine, ultrasound, elastography, photoacoustic imaging, tomography, functional near-infrared spectroscopy, and magnetic particle imaging.

The term “endocytosis” encompasses (1) phagocytosis and (2) pinocytosis, itself a category inclusive of (2a) macropinocytosis, which does not require receptor binding, as well as of (2b) clathrin-mediated endocytosis, (2c) caveolae-mediated endocytosis and (2d) clathrin-/caveolae-independent endocytosis, all of which tend to access the late-endosome/lysosome pathway.

“Sequence identity” refers to “percent (%) nucleic acid or amino acid sequence identity” when a first polypeptide is being compared with a second polypeptide or a first polynucleotide is being compared with a second polynucleotide. The phrase refers to the percentage of nucleotide or amino acid residues in a first sequence that are identical with the nucleotide or amino acid residues in a second sequence. The sequence identity values between two polypeptides or two polynucleotides may be determined by the BLASTN module of WU-BLAST-2 set to the default parameters.

As noted, the minicell compositions of the present disclosure are useful in delivering anti-neoplastic agents to the tumors. In this context, the phrase “anti-neoplastic agent” denotes a drug, whether chemical or biological, that prevents or inhibits the growth, development, maturation, or spread of neoplastic or tumor cells.

The following examples are provided to illustrate the present invention. It should be understood, however, that the invention is not to be limited to the specific conditions or details described in these examples. Throughout the specification, any and all references to a publicly available document, including a U.S. and/or international patent or patent application publication, are specifically incorporated by reference

### EXAMPLES

### Example 1: Preparation of Antineoplastic Packaged Minicells

Minicells may be produced and purified from various bacterial strains, including for example a Salmonella enterica serovar Typhimurium (S. Typhimurium) minCDE-strain as previously described (MacDiarmid et al., 2007b). Anti-neoplastic agent loading, antibody targeting, lyophilization, and dose preparation have been previously described (MacDiarmid et al., 2007b; Sagnella et al., 2018).

Minicell preparations may be subject to strict quality control in which minicell size and number are assessed using dynamic light scattering using a Zetasizer Nano Series and NanoSight LM20 (Malvern Instrument). Antineoplastic drug may be extracted from minicell preparations and quantified via IPLC as previously described (MacDiarmid et al., 2007b).

### Example 2: Culture of Target Cancer Cells for Testing Minicells

RAW264.7 cells (ATCC) may be grown to ˜70% confluence in Dulbecco's Modified Eagle Media (DMEM) (Sigma) containing 10% FCS and passaged using a cell scraper. Mouse tumor cell lines (4T1 and CT26) may be grown in monolayers in RPMI-1640 media (Sigma) containing 10% FCS and passaged 2-3 times per week using phosphate buffered saline (PBS)/Trypsin EDTA.

All cells may be maintained in culture at 37° C. in a humidified atmosphere containing 5% CO₂ and should be routinely screened and found to be free of mycoplasma. EpCAM expression and receptor number in the mouse cell lines may be quantified using flow cytometry with APC anti-mouse CD326 (Biolegend) using Quantum Simply Cellular anti-Rat IgG microspheres (Bangs Laboratory). CT26 may be shown to be negative for EpCAM, cells should then be transfected with a pcDNA3.1+C DYK containing the mouse EpCAM ORF clone (NM_008532.2) (Genescript) using Lipofectamine 2000 (Thermo Fisher). G418 selection is used to obtain pure populations of EpCAM expressing CT26 clones, and cells are screened as described above for EpCAM expression.

Clones are examined for growth rate, drug sensitivity and in vivo tumorgenicity, and a clone that possesses high EpCAM expression with the above 3 parameters being similar to the parental CT26 cell line may be selected for all subsequent studies (CT26Ep12.1).

Bone marrow derived DCs (BMDCs) may be prepared as follows. Bone marrow may be isolated from the femurs and tibias of Balb/c mice. Following red blood cell lysis and washing, cells may be resuspended in AIMV+5% FBS+2-mercaptoethanol+penicillin/streptomycin+20 ng/ml GM-CSF (Miltenyi Biotec) and grown for 7 days.

### Example 3: Treatment of RAW264.7 Cells with Minicells (EDVs)

RAW264.7 cells may be seeded in 6-well plates at 3×10⁵ cells per well and incubated overnight. Media should then replaced with fresh media containing one of the following: 1 μg/mL LPS (Sigma); 100 pmol PNU-159682 (Najing Levena); Ep-EDV-682 (500:1 and 1000:1 EDV: cells), Ep-EDV (5000:1 EDV:cells), or left untreated. Cells may be harvested 6 h and 24 h post treatment using a cell scraper and samples should be stained with DAPI (Sigma), anti-CD45 Brilliant Violet 510 (BioLegend), anti-CD86 APC-Cy7 (BioLegend), and anti-CD206 AF488 (R&D Systems) and assessed by flow cytometry.

### Example 4: Macrophage and DC/Tumor Cell Co-Cultures

CT26Ep12.1 and 4T1 cells may be harvested with Versene (Gibco) and cells collected in 1 mL Eppendorf tubes. Cells may be resuspended in 1 mL DMEM (Sigma) supplemented with 10% FBS (Bovogen) containing: minicells without anti-neoplastic and radioimaging agent, minicells with anti-neoplastic agent, mincells with radioimaging agent, and minicells with anti-neoplastic agent and radioimaging agent.

Anti-neoplastic agent, radioimaging agent and minicell amounts may be established via MTS and XCELLigence real time experiments such that chosen concentrations result in the initiation of cell death within the first 24 h post treatment. Cells are then washed thoroughly with PBS to remove any non-adherent EDV or excess drug. Treated tumor cells may be cultured overnight with either RAW264.7 or BMDC at a 1:1 ratio of tumor cells: RAW264.7/BMDC/JAWS II. Supernatants are collected for ELISA analysis. RAW264.7/tumor cell co-cultures are collected using a cell scraper and samples are stained with DAPI (Sigma), anti-CD45 Brilliant Violet 510 (BioLegend), anti-CD86 APC-Cy7, and anti-CD206 AF488 and assessed by flow cytometry. JAWS II/tumor cell and BMDC/tumor cell co-cultures are collected with versene and stained with DAPI (Sigma), CD11b AF488 (Abcam), CD11c PE (Molecular Probes), anti-CD45 Brilliant Violet 510 or PECy5 (BioLegend), anti-CD86 APC-Cy7, MHC Class II PECy5 (Thermo Fisher), MHC Class II Brilliant Violet 421 (BioLegend), 7-AAD (BioLegend), and/or CD80 PE (Thermo Fisher) and assessed by flow cytometry. RNA is extracted from BMDC/tumor cell co-cultures using an RNAeasy Plus Mini Kit (Qiagen) according to the manufacturer's protocol.

Cells may be lysed and homogenized in RLT buffer, and passed through a gDNA eliminator spin column. 70% ethanol may be added to the flow through and samples are then passed through an RNeasy spin column, washed and eluted in RNase-free water. RNA concentration may be determined on an Eppendorf biophotometer plus. The RNA is used to reverse transcribe cDNA using a SuperScript™ VILO™ cDNA Synthesis Kit (Thermo Fisher) according to the manufacturer's protocol. The transcribed cDNA should be diluted 1:2 for qPCR. Each qPCR reaction may 5 uL TaqMan fast advanced master mix (Thermo Fisher), 0.5 uL 20× Taqman primer/probe mix (IFNα Mm03030145_gH, IFNb1 Mm00439552_s1, GAPDH Mm99999915_g1; Thermo Fisher) and 2.5 uL of water. 8 μL of the mix plus 2 μL of cDNA should be added into a 96 well plate. qPCR may be performed using an Applied Biosystems Real-Time PCR System. Data may be exported to excel and the relative quantitation calculated from the ΔΔCt.

### Example 5: In Vivo Tumor Models

For the 4T1 and CT26Ep12.1 model, female BALB/c mice may be obtained from Animal Resources Centre at 6-8 weeks of age. For T84 and A549/MDR models BALB/c Fox1^{nu}/ARC may be obtained from Animal Resources Centre at 5-7 weeks of age. After at least 1 week of observation, mice may be injected with 5×10⁴ 4T1 cells per 50 μl PBS into the 3rd mammary fat pad on the right hand side or 2×10⁵ CT26Ep12.1 per 100 μl PBS subcutaneously into the right flank of BALB/c mice. For human xenografts, 5×10⁶ A549/MDR or 1×10⁷ T84 per 100 μl PBS/Matrigel (Sigma) may be subcutaneously injected into the right flank.

Treatment may be commenced on day 7 post tumor induction for the 4T1 model, when the average tumor size is ˜90 mm³, and on day 9 for the CT26Ep12.1 model when the average tumor size is ˜125 mm³. Mice may be treated via i.v, tail vein injection three times weekly for 2 weeks with one of the following treatments: Saline, 1×10⁹ EpCAM targeted minicells, 1×10⁹ EpCAM targeted minicells loaded with antineoplastic, for example, PNU-159682, 1×10⁹ EpCAM targeted minicells conjugated with radioimaging agent, or 1×10⁹ EpCAM targeted minicells conjugated with radioimaging agent and loaded with antineoplastic. Tumors may be measured 3 times/week and tumor volume may be calculated as π/6(Length×Width×Height). At the end of the 2 week period, mice may be humanely euthanized and tumors and spleens collected for ex vivo analysis.

Treatment of A549/MDR and T84 tumors may be commenced when tumors reach 100-120 mm³ and 120-150 mm³ respectively. Mice may be treated with Saline, 1×10⁹ EGFR targeted minicells loaded with the antineoplastic Doxorubicin, 1×10⁹ EGFR targeted minicells conjugated with radioimaging agent, 1×10⁹ EGFR targeted minicells loaded with the antineoplastic Doxorubicin and conjugated with radioimaging agent, 1×10⁹ EGFR targeted minicells loaded with PNU-159682, 1×10⁹ EGFR targeted minicells conjugated with radioimaging agent, 1×10⁹ EGFR targeted minicells loaded with PNU-159682 and conjugated with radioimaging agent, 1×10⁹ non-targeted minicells loaded with antineoplastic PNU-159682 (EDV-682), 1×10⁹ non-targeted minicells conjugated with radioimaging agent, 1×10⁹ non-targeted minicells loaded with antineoplastic PNU-159682 (EDV-682) and conjugated with radioimaging agent.

### Example 6: Isolation of CD11b⁺ Cells from 4T1 and CT26Ep12.1 Tumors

Tumors may be dissected, weighed, and enzymatically digested using a Tissue Dissociation Kit (Miltenyi Biotec) at 37° C. according to the manufacturer's instructions, using the gentleMACS™ Dissociator. Following dissociation, red blood cells may be removed using RBC lysis buffer (Sigma). After washing, cells may be passed through a 70 μm cell strainer to remove any clumps. CD11b⁺ cells may be purified by positive selection using CD11b MACS beads (Miltenyi Biotec) on LS column on the MACS separator (Miltenyi Biotec). The purity of the isolated CD11b⁺ cell population may be assessed by flow-cytometry with an APC anti-mouse CD11b (Biolegend).

### Example 7: Isolation of NK and CD8 from Spleens

Spleens may be homogenized using a Dounce homogenizer and filtered through 70 μM mesh strainers to obtain single cell suspension followed by erythrocyte lysis using RBC lysis buffer. Splenocytes may be washed and a cell count performed before progressing to NK or CD8+ T-cell isolation. NK cells and CD8+ T cells may be isolated from dissociated spleen cells by negative selection using either the NK Cell Isolation II kit (Miltenyi Biotec) or the CD8a+ T Cell Isolation Kit (Miltenyi Biotec), according to the manufacturer's instructions. Cells may be separated by using an LS column on the MACS separator (Miltenyi Biotec). NK cell and CD8+ T-cell preparations may be assessed by flow-cytometry. NK cells may be rested overnight in RPMI-1640 media supplemented with 10% FBS at 37° C. prior to the NK cell-mediated cytolysis assay. CD8+ T-cells may be added to tumor cells immediately following isolation to assess CD8+ T-cell cytolysis.

### Example 8: Isolation of NK and CD8 from Spleens

Spleens may be homogenized using a Dounce homogenizer and filtered through 70 μM mesh strainers to obtain single cell suspension followed by erythrocyte lysis using RBC lysis buffer. Splenocytes may be washed and a cell count performed before progressing to NK or CD8+ T-cell isolation. NK cells and CD8+ T cells may be isolated from dissociated spleen cells by negative selection using either the NK Cell Isolation II kit (Miltenyi Biotec) or the CD8a+ T Cell Isolation Kit (Miltenyi Biotec), according to the manufacturer's instructions. Cells may be separated by using an LS column on the MACS separator (Miltenyi Biotec). NK cell and CD8+ T-cell preparations may be assessed by flow-cytometry. NK cells may be rested overnight in RPMI-1640 media supplemented with 10% FBS at 37° C. prior to the NK cell-mediated cytolysis assay. CD8+ T-cells may be added to tumor cells immediately following isolation to assess CD8+ T-cell cytolysis.

### Example 9: XCELLigence Monitored CD11b+, CD8+, and NK Cell Cytolysis of Tumor Cells

Cell growth characteristics and tumor cell death may be monitored in real time by the xCELLigence DP system. To do so, circular electrodes covering the base of the tissue culture wells detect changes in electrical impedance and convert the impedance values to a Cell Index (CI). Cell Index measurements directly correspond to the strength of cell adhesion and cell number. Target cells (4T1, CT26Ep12.1, A549/MDR, or T84) were seeded into an E-Plate 16. Cells may be allowed to attach and proliferate till they have reached their logarithmic growth phase. The effector cells (CD11b+ cells, NK cells, or CD8+ T-cells) may be added to the target cells at the following effector-to-target cell ratios: 5:1 (CD11b+: tumor cell), 20:1 (NK cell: mouse tumor cell), 10:1 (NK:human tumor cell), and 30:1 (CD8+ T-cell: tumor cell). After addition of effector cells, the system may take regular measurements (every 5 or 15 min) for 3-4 days to monitor immune cell-mediated killing of tumor cells.

### Example 10: NK Cell Mediated Cytolysis Inhibition

Mouse tumor cell lines are initially screened for NK cell ligand expression via flow cytometry with anti-Rae-1α/β/γ-PE (Miltenyi Biotec), anti-H60a-PE (Miltenyi Biotec), and anti-MULT-1 PE (R&D Systems). For NK cell-mediated cytolysis inhibition based on these ligand expression levels, the effector NK cells may be added to target cells in the presence of 3 μg/ml of blocking mAb to the following NK cell ligands: anti-RAE-1αβγ (R&D Systems) or anti-H60 (R&D Systems) separately and as mixture. xCELLigence data may be transformed in Excel and exported to Prism (GraphPad Software) for graphing and statistical analysis.

### Example 11: Tumor/Spleen Flow Cytometry

Tumors and spleens may be dissociated as described above. Following red blood cell lysis, cells may be incubated with Fc block 1:10 in MACS buffer (Miltenyi Biotec) for 10 min. After the 10 min incubation, cells may be washed once and incubated with a primary antibody panel in MACS buffer for 15 min on ice in the dark. Cells may be washed 2 times and then resuspended in MACS buffer for flow cytometric analysis. The following antibodies may be used in T-cell, NK cell, and macrophage staining panels: anti-CD45 PECy7 (BioLegend), anti-CD45 BV510 (Biolegend), anti-CD3e APC-eFluor780 (eBioscience), anti-CD3 APC (Molecular Probes), anti-CD4 PE-TR (Abcam), anti-CD8a FITC (eBioscience), anti-CD8 BV510 (BioLegend), anti-CD25 PE (Abcam), anti-CD314 (NKG2D) PE-eFluor610 (eBioscience), anti-CD335 (NKp46) PECy7 (BioLegend), anti-CD27 BV421 (BioLegend), ant-CD183 (CXCR3) BV510 (BioLegend), anti-NKG2A/C/E FITC (eBioscience), anti-CD11b APC (BD Pharmingen), anti-Ly6C FITC (BioLegend), anti-Ly6G BV510 (BioLegend), anti-F4/80 PE Dazzle594 (BioLegend), anti-CD206 PECy7 (BioLegend), and anti-CD86 APC-Cy7 (BioLegend). Single stained controls and/or versacomp (Beckman Coulter) beads may be used for fluorescence compensation. DAPI (Sigma), propidium iodide (Sigma), DRAQ5 (Thermo Fisher), or Live/Dead Yellow (Thermo Fisher) may be used for live cell detection. Unstained and isotype controls may be employed to determine auto-fluorescence levels and confirm antibody specificity.

### Example 12 Confocal Microscopy

4T1 cells may be seeded on Lab-Tek chamber slides (Sigma) and left to attach and grow for 24 h. Isolated CD8+ T-cells may be added to the 4T1 cells and left for 8 h, at which time, cells may be fixed in 4% paraformaldehyde. Cells may be washed and permeabilized with 0.5% triton-x-100 in PBS (PBST). Cells may be blocked with 3% BSA for 30 min followed by incubation with the primary anti-perforin antibody (Abcam) diluted in PBST. After washing, cells may be incubated with the secondary goat anti-rat IgG Alexafluor 488 (Abcam), followed by incubation with AlexaFluor 568 Phalloidin (Thermo Fisher). Cells may be mounted with Prolong Diamond Antifade with DAPI (Thermo Fisher) and sealed with nail polish prior to imaging. Images may be acquired on a Zeiss LSM 780, and images were merged and processed in Image J.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof, inclusive of the endpoints. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

### REFERENCES

Mango L, Pacilio M. Therapy with Alpha Rays. ARC Journal of Radiology and Medical Imaging. 2016; 1: 1-3.
Sun H, Zu Y. Aptamers and their applications in nanomedicine. Small 2015; 11: 2352-64.
Lao et al., “Aptamer nanomedicine for cancer therapeutics: barriers and potential for translation,” ACS Nano 2015; 9: 2235-54.
Zhang et al., “Polyelectrolyte Multilayers Assembled Entirely from Immune Signals on Gold Nanoparticle Templates Promote Antigen-Specific T Cell Response,” ACS Nano 2015; 9: 6465-77.
Zhao et al., “Phage antibody display libraries: a powerful antibody discovery platform for immunotherapy,” Crit Rev Biotechnol 2016; 36: 276-89.
Tohidkia M R, Barar J, Asadi F, Omidi Y. Molecular considerations for development of phage antibody libraries. J Drug Target 2012; 20: 195-208.
Lin et al., “Drug/Dye-Loaded, Multifunctional PEG-Chitosan-Iron Oxide Nanocomposites for Methotraxate Synergistically Self-Targeted Cancer Therapy and Dual Model Imaging,” ACS Appl Mater Interfaces 2015; 7: 11908-20.
Heidari et al., “Tamoxifen loaded folic acid armed PEGylated magnetic nanoparticles for targeted imaging and therapy of cancer,” Colloids Surf B Biointerfaces 2013; 106: 117-25.
Omidi Y, Barar J. Induction of human alveolar epithelial cell growth factor receptors by dendrimeric nanostructures. Int J Toxicol 2009; 28: 113-22.
Hartono et al., “Poly-L-lysine functionalized large pore cubic mesostructured silica nanoparticles as biocompatible carriers for gene delivery,” ACS Nano 2012; 6: 2104-17.
Ezzati Nazhad Dolatabadi J, Omidi Y. Solid lipid-based nanocarriers as efficient targeted drug and gene delivery systems. TrAC Trends in Analytical Chemistry 2016; 77: 100-8.
Majkowska et al., “Complexes of low energy beta emitters 47Sc and 177Lu with zoledronic acid for bone pain therapy,” Appl Radiat Isot. 2009; 67(1):11-13.
Palmedo H. Radionuclide therapy of bone metastases. In: Biersack H J, Freeman L M, editors. Clinical Nuclear Medicine. Berlin, Heidelberg: Springer Berlin Heidelberg; 2007:433-442.
Pillai et al., “Production logistics of 177Lu for radionuclide therapy,” Appl Radiat Isot. 2003; 59(2-3):109-118.
Ahmadzadehfar et al., “Radioembolization of liver tumors with yttrium-90 microspheres,” Semin Nucl Med. 2010; 40(2):105-121.
Teunissen et al., “Endocrine tumours of the gastrointestinal tract. Peptide receptor radionuclide therapy,” Best Pract Res Clin Gastroenterol. 2005; 19(4):595-616.
Ahmadzadehfar et al., “Therapeutic response and side effects of repeated radioligand therapy with 177Lu-PSMA-DKFZ-617 of castrate-resistant metastatic prostate cancer,” Oncotarget. 2016; 7(11):12477-12488.
Kwekkeboom D J, de Herder W W, Kam B L, et al. Treatment with the radiolabeled somatostatin analog [177 Lu-DOTA 0, Tyr3]octreotate: toxicity, efficacy, and survival. J Clin Oncol. 2008; 26(13):2124-2130.
Kramer-Marek G, Capala J. The role of nuclear medicine in modern therapy of cancer. Tumour Biol. 2012; 33(3):629-640.
Santoni M, Scarpelli M, Mazzucchelli R, et al. Targeting prostate-specific membrane antigen for personalized therapies in prostate cancer: morphologic and molecular backgrounds and future promises. J Biol Regul Homeost Agents. 2014; 28(4):555-563.
Silver D A, Pellicer I, Fair W R, Heston W D, Cordon-Cardo C. Prostate-specific membrane antigen expression in normal and malignant human tissues. Clin Cancer Res. 1997; 3(1):81-85.
Ghosh A, Heston W D W. Tumor target prostate specific membrane antigen (PSMA) and its regulation in prostate cancer. J Cell Biochem. 2004; 91(3):528-539.
Mhawech-Fauceglia P, Zhang S, Terracciano L, et al. Prostate-specific membrane antigen (PSMA) protein expression in normal and neoplastic tissues and its sensitivity and specificity in prostate adenocarcinoma: an immunohistochemical study using multiple tumour tissue microarray technique. Histopathology. 2007; 50(4):472-483.
Eckelman W C, Gibson R E. The design of site-directed radiopharmaceuticals for use in drug discovery. In: Nuclear Imaging In Drug Discovery, Development, and Approval: Springer; 1993:113-134.
Holman B L, Tumeh S S. Single-photon emission computed tomography (SPECT): applications and potential. JAMA. 1990; 263(4):561-564.
Gerard S K, Cavalieri R R. I-123 diagnostic thyroid tumor whole-body scanning with imaging at 6, 24, and 48 hours. Clin Nucl Med. 2002; 27(1):1-8.
Alzahrani A S, AlShaikh O, Tuli M, Al-Sugair A, Alamawi R, Al-Rasheed M M. Diagnostic value of recombinant human thyrotropin-stimulated ¹²³I whole-body scintigraphy in the follow-up of patients with differentiated thyroid cancer. Clin Nucl Med. 2012; 37(3):229-234.
Walrand S, Hesse M, Renaud L, Jamar F. The impact of image reconstruction bias on PET/CT ⁹⁰Y dosimetry after radioembolization. J Nucl Med. 2015; 56(3):494-495.
Müller C, Zhernosekov K, Köster U, et al. A unique matched quadruplet of terbium radioisotopes for PET and SPECT and for alpha- and beta-radionuclide therapy: an in vivo proof-of-concept study with a new receptor-targeted folate derivative. J Nucl Med. 2012; 53(12):1951-1959.
Lee S, Xie J, Chen X. Peptide-based probes for targeted molecular imaging. Biochemistry. 2010; 49:1364-1376.
Roberts M, Bentley M, Harris J. Chemistry for peptide and protein PEGylation. Adv Drug Deliv Rev. 2012; 64:116-127.
Chatalic K L S, Kwekkeboom D J, de Jong M. Radiopeptides for imaging and therapy: a radiant future. J Nucl Med. 2015; 56:1809-1812.
Erathodiyil N, Ying J Y. Functionalization of inorganic nanoparticles for bioimaging applications. Acc Chem Res. 2011; 44:925-935. [PubMed: 21648430]
Sosabowski J K, Mather S J. Conjugation of DOTA-like chelating agents to peptides and radiolabeling with trivalent metallic isotopes. Nat Protoc. 2006; 1:972-976.
Sun X, Li Y, Liu T, Li Z, Zhang X, Chen X. Peptide based imaging agents for cancer detection. Adv Drug Deliv Rev. 110-111: 38-51 (2017).
Takahashi H, Emoto K, Dubey M, Castner D G, Grainger D W. Imaging surface immobilization chemistry: correlation with cell patterning on non-adhesive hydrogel thin films. Adv Funct Mater. 2008; 18:2079-2088.
Chen K, Chen X. Design and development of molecular imaging probes. Curr Top Med Chem. 2010; 10:1227.
De Jong M, Breeman W A, Kwekkeboom D J, Valkema R, Krenning E P. Tumor imaging and therapy using radiolabeled somatostatin analogues. Acc Chem Res. 2009; 42:873-880.
Galluzzi L, Kepp O, Vander Heiden M G, Kroemer G. Metabolic targets for cancer therapy. Nat Rev Drug Discov. 2013; 12:829-846.
Hoelder S, Clarke P A, Workman P. Discovery of small molecule cancer drugs: successes, challenges and opportunities. Mol Oncol. 2012; 6:155-176.
Hong H, Goel S, Zhang Y, Cai W. Molecular imaging with nucleic acid aptamers. Curr Med Chem. 2011; 18:4195.
Igarashi H, Fujimori N, Ito T, Nakamura T, Oono T, Nakamura K, Suzuki K, Jensen R T, Takayanagi R. Vasoactive intestinal peptide (VIP) and VIP receptors—elucidation of structure and function for therapeutic applications. Int J Clin Med. 2011; 2:500-508.
James M L, Gambhir S S. A molecular imaging primer: modalities, imaging agents, and applications. Physiol Rev. 2012; 92:897-965.
Laverman P, Sosabowski J K, Boerman O C, Oyen W J. Radiolabelled peptides for oncological diagnosis. Eur J Nucl Med Mol Imaging. 2012; 39:78-92.
Lin V S, Chen W, Xian M, Chang C J. Chemical probes for molecular imaging and detection of hydrogen sulfide and reactive sulfur species in biological systems. Chem Soc Rev. 2015; 44:4596-4618.
Schottelius M, Wester H-J. Molecular imaging targeting peptide receptors. Methods. 2009; 48:161-177.
Tweedle M F. Peptide-targeted diagnostics and radiotherapeutics. Acc Chem Res. 2009; 42:958-968.
Andersson L, Blomberg L, Flegel M, Lepsa L, Nilsson B, Verlander M. Large-scale synthesis of peptides. Pept Sci. 2000; 55:227-250.
Deutscher S L. Phage display in molecular imaging and diagnosis of cancer. Chem Rev. 2010; 110:3196-3211.
Gray B P, Brown K C. Combinatorial peptide libraries: mining for cell-binding peptides. Chem Rev. 2013; 114:1020-1081.
Merrifield R. Solid-phase peptide synthesis. Adv Enzymol Relat Areas Mol Biol. 2006; 32:221-296.
Nagpal R, Behare P, Rana R, Kumar A, Kumar M, Arora S, Morotta F, Jain S, Yadav H. Bioactive peptides derived from milk proteins and their health beneficial potentials: an update. Food Funct. 2011; 2:18-27.
Burger M, Hartmann T, Krome M, Rawluk J, Tamamura H, Fujii N, Kipps T J, Burger J A. Small peptide inhibitors of the CXCR4 chemokine receptor (CD184) antagonize the activation, migration, and antiapoptotic responses of CXCL12 in chronic lymphocytic leukemia B cells. Blood. 2005; 106:1824-1830.
Igarashi H, Fujimori N, Ito T, Nakamura T, Oono T, Nakamura K, Suzuki K, Jensen R T, Takayanagi R. Vasoactive intestinal peptide (VIP) and VIP receptors—elucidation of structure and function for therapeutic applications. Int J Clin Med. 2011; 2:500-508.
Matsuno M, Matsui T, Iwasaki A, Arakawa Y. Role of acetylcholine and gastrin-releasing peptide (GRP) in gastrin secretion. J Gastroenterol. 1997; 32:579-586.
Ohki-Hamazaki H, Iwabuchi M, Maekawa F. Development and function of bombesin-like peptides and their receptors. Int J Dev Biol. 2005; 49:293-300.
Ruoslahti E. RGD and other recognition sequences for integrins. Annu Rev Cell Dev Biol. 1996; 12:697-715.
Singh M, Mukhopadhyay K. Alpha-melanocyte stimulating hormone: an emerging anti-inflammatory antimicrobial peptide. Biomed Res Int. 2014; 2014:874610.
Tyler-McMahon B M, Boules M, Richelson E. Neurotensin: peptide for the next millennium. Regul Pept. 2000; 93:125-136.
Strand, F L. Neuropeptides: Regulators of Physiological Processes. MIT press; 1999.
Tatemoto, K. Neuropeptide Y: history and overview, Neuropeptide Y and Related Peptides. Springer; 2004. p. 1-21.
Weckbecker G, Lewis I, Albert R, Schmid H A, Hoyer D, Bruns C. Opportunities in somatostatin research: biological, chemical and therapeutic aspects. Nat Rev Drug Discov. 2003; 2:999-1017.
Yang W, Luo D, Wang S, Wang R, Chen R, Liu Y, Zhu T, Ma X, Liu R, Xu G. TMTP1, a novel tumor-homing peptide specifically targeting metastasis. Clin Cancer Res. 2008; 14:5494-5502.
Cheng Z, Wu Y, Xiong Z, Gambhir S S, Chen X. Near-infrared fluorescent RGD peptides for optical imaging of integrin αvβ3 expression in living mice. Bioconjug Chem. 2005; 16:1433-1441.
Zhu L, Guo N, Li Q, Ma Y, Jacboson O, Lee S, Choi H S, Mansfield J R, Niu G, Chen X. Dynamic PET and optical imaging and compartment modeling using a dual-labeled cyclic RGD peptide probe. Theranostics. 2012; 2:746-756.
Chen H W, Wang L Y, Yu Q Q et al. Anti-HER2 antibody and ScFvEGFR-conjugated antifouling magnetic iron oxide nanoparticles for targeting and magnetic resonance imaging of breast cancer. Int. J. Nanomed. 8, 3781-3794 (2013).
Chen H W, Wang L Y, Yeh J et al. Reducing non-specific binding and uptake of nanoparticles and improving cell targeting with an antifouling PEO-b-P gamma MPS copolymer coating. Biomaterials 31(20), 5397-5407 (2010).
Lee H, Lee E, Kim D K, Jang N K, Jeong Y Y, Jon S. Antibiofouling polymer-coated superparamagnetic iron oxide nanoparticles as potential magnetic resonance contrast agents for in vivo cancer imaging. J. Am. Chem. Soc. 128(22), 7383-7389 (2006).
Park S J, Lee K B, Choi I S, Langer R, Jon S Y. Dual functional, polymeric self-assembled monolayers as a facile platform for construction of patterns of biomolecules. Langmuir 23(22), 10902-10905 (2007).
Markman J L, Rekechenetskiy A, Holler E, Ljubimova J Y. Nanomedicine therapeutic approaches to overcome cancer drug resistance. Adv. Drug Deliv. Rev. 65(14), 1866-1879 (2013).
Boni A, Marinone M, Innocenti C et al. Magnetic and relaxometric properties of Mn ferrites. J. Phys. D AppL. Phys. 41(13), 134021 (2008).
Huang J, Wang L Y, Zhong X D, Li Y C, Yang L L, Mao H. Facile non-hydrothermal synthesis of oligosaccharide coated sub-5 nm magnetic iron oxide nanoparticles with dual MRI contrast enhancement effects. J. Mater. Chem. B 2(33), 5344-5351 (2014).
Huang J, Li Y C, Orza A et al. Magnetic nanoparticle facilitated drug delivery for cancer therapy with targeted and image-guided approaches. Adv. Funct. Mater. 26(22), 3818-3836 (2016).
Laurent S, Forge D, Port M et al. Magnetic iron oxide nanoparticles: synthesis, stabilization, vectorization, physicochemical characterizations, and biological applications. Chem. Rev. 108(6), 2064-2110 (2008).
Laurent S, Boutry S, Mahieu I, Vander Elst L, Muller R N. Iron oxide based M R contrast agents: from chemistry to cell labeling. Curr. Med. Chem. 16(35), 4712-4727 (2009).
Xie J, Chen K, Lee H Y et al. Ultrasmall c(RGDyK)-coated Fe₃O₄ nanoparticles and their specific targeting to integrin alpha(v)beta3-rich tumor cells. J. Am. Chem. Soc. 130(24), 7542-7543 (2008).
Sun S H, Zeng H. Size-controlled synthesis of magnetite nanoparticles. J. Am. Chem. Soc. 124(28), 8204-8205 (2002).
Wang L N, Tang W, Zhen Z P, Chen H M, Xie J, Zhao Q. Improving detection specificity of iron oxide nanoparticles (IONPs) using the SWIFT sequence with long T-2 suppression. Magn. Reson. Imaging 32(6), 671-678 (2014).
Kojima H, Mukai Y, Yoshikawa M, Kamei K, Yoshikawa T, Morita M, et al. Simple PEG conjugation of SPIO via an Au—S bond improves its tumor targeting potency as a novel M R tumor imaging agent. Bioconjug Chem 2010; 21: 1026-31.
Cabana L, Bourgognon M, Wang J T, Protti A, Klippstein R, de Rosales R T, et al. The Shortening of MWNT-SPION Hybrids by Steam Treatment Improves Their Magnetic Resonance Imaging Properties In Vitro and In Vivo. Small 2016; 12: 2893-905.
Frangville C, Li Y, Billotey C, Talham D R, Taleb J, Roux P, et al. Assembly of Double-Hydrophilic Block Copolymers Triggered by Gadolinium Ions: New Colloidal MRI Contrast Agents. Nano Lett 2016; 16: 4069-73.
de Barros et al. EJNMMI Res (2012) 2: 39.
Liu J, Li Z, Yang X, Liu W, Wang B, Zhu Y, et al. A high-performance imaging probe with NIR luminescence and synergistically enhanced T1-T2 relaxivity for in vivo hepatic tumor targeting and multimodal imaging. Chem Commun (Camb) 2015; 51: 13369-72.

1. A theranostic composition comprising:
(a) a plurality of purified, intact bacterially derived minicells;
(b) at least one anti-neoplastic agent comprised within the minicells;
(c) a bispecific ligand, wherein the bispecific ligand is attached to a first surface component of the minicells, and wherein the bispecific ligand comprises:
(i) a first arm with binding specificity for the first surface component; and
(ii) a second arm with binding specificity for a tumor cell surface receptor; and
(d) a monospecific ligand attached to a second surface component of the minicells, wherein the monospecific ligand has at least one radio-imaging agent conjugated to the monospecific ligand at one or more conjugation residues of the monospecific ligand.

2. The theranostic composition of claim 1, wherein the one or more conjugation residues is each independently selected from the group consisting of an F-amino group on a lysine side chain, a guanidinium group on an arginine side chain, a carboxyl group on an aspartic acid or glutamic acid, a cysteine thiol, a phenol on a tyrosine, and a combination thereof.

3. The theranostic composition of claim 1, wherein:
(a) the bispecific ligand and/or the monospecific ligand comprises a polypeptide, aptamer, carbohydrate, or a combination thereof, and/or
(b) the bispecific ligand has a specificity to a non-phagocytic mammalian tumor cell surface receptor; and/or
(c) the bispecific ligand comprises an antibody that specifically recognizes the tumor cell antigen; and/or
(d) the monospecific ligand has a variable length and comprises a polypeptide comprising from about 15 to about 500 amino acids; and/or
(e) the monospecific ligand can be increased in length to produce a corresponding increase in an amount of the radio-imaging agent.

4. The theranostic composition of claim 1, wherein:
(a) the amount of radio-imaging agent conjugated to the monospecific ligand varies directly with the amount of conjugation residues; and/or
(b) the composition comprises a diagnostically effective amount of radio-imaging agent sufficient to produce a clear image of the tumor upon radioimaging; and/or
(c) the radio-imaging agent also functions as a therapeutic radiation emitting agent, and wherein the amount of radiation emitted by the radio-imaging agent is sufficient to provide a therapeutic effect on the tumor, and optionally wherein the therapeutic effect is a reduction in tumor size; and/or optionally wherein the tumor is reduced in size by about 100%, about 90%, about 80%, about 70%, about 60%, about 50%, about 40%, about 30%, about 20%, about 10%, or about 5%.

5. The theranostic composition of claim 1:
(a) comprising about 30 Gy to about 100 Gy radiation; and/or
(b) wherein the minicell has a diameter of about 100 nm to about 600 nm; and/or
(c) wherein the minicell is PEGylated.

6. The theranostic composition of claim 1, wherein the radio-imaging agent:
(a) comprises a radioisotope, magnetic nanoparticle, organic fluorescent dye, or any combination thereof, and/or
(b) comprises a radioisotope selected from the group consisting of yttrium-90, yttrium-86, terbium-152, terbium-155, terbium-149, terbium-161, technetium-99m, iodine-123, iodine-131, rubidium-82, thallium-201, gallium-67, fluorine-18, copper-64, gallium-68, xenon-133, indium-111, lutetium-177, and any combination thereof, and/or
(c) is comprised within a synthetic nanoparticle, and wherein the synthetic nanoparticle is conjugated to the monospecific ligand; and/or
(d) is conjugated to the monospecific ligand via a linker.

7. The theranostic composition of claim 1, wherein:
(a) the minicell comprises a pharmaceutically effective polymer film or coat;
(b) the minicell comprises a pharmaceutically effective polymer film or coat, wherein the polymer film or coat is opsonization-reducing; and/or
(c) the minicell comprises a pharmaceutically effective polymer film or coat, wherein the polymer film or coat reduces or minimizes macrophage uptake of the composition; and/or
(d) the polymer film or coat comprises a polymer selected from the group consisting of a polyethylene glycol, polymer-PEO-blockpoly(γ-methacryloxypropyltrimethoxysilane) (PEOb-PγMPS), and (trimethoxysilyl)propyl methacrylate-PEG-methacrylate.

8. The theranostic composition of claim 1, wherein the tumor cell surface receptor:
(a) comprises a tumor cell antigen; and/or
(b) comprises an integrin, neuromedin B receptor, bombesin 3 receptor, GRP receptor, bombesin 4 receptor, CCK2/gastrin, melanocortin-1 receptor (MC-1r), neuropeptide Y (NPY) receptor, neutrotensin (NT) receptor, prostate specific membrane antigen (PSMA), somatostatin (SST) receptor, neurokinin 1 receptor (NK1R), chemokine receptor type 4 (CXCR4), vasoactive intestinal peptide (VIP), epidermal growth factor receptor (EGFR), vascular endothelial growth factor receptor (VEGFR), platelet-derived growth factor receptor (PDGFR), insulin-like growth factor receptor (IGFR), or any combination thereof, and/or
(c) comprises EpCAM, CCR5, CD19, HER-2 neu, HER-3, HER-4, EGFR, PSMA, CEA, MUC-1 (mucin), MUC2, MUC3, MUC4, MUC5, MUC5, MUC7, BhcG, Lewis-Y, CD20, CD33, CD30, ganglioside GD3, 9-O-Acetyl-GD3, GM2, Globo H, fucosyl GM1, Poly SA, GD2, carboanhydrase IX (MN/CA IX), CD44v6, sonic hedgehog (Shh), Wue-1, Plasma Cell Antigen, (membrane-bound) IgE, melanoma chondroitin sulfate proteoglycan (MCSP), CCR8, TNF-alpha precursor, STEAP, mesothelin, A33 antigen, prostate stem cell antigen (PSCA), Ly-6; desmoglein 4, E-cadherin neoepitope, fetal acetylcholine receptor, CD25, CA19-9 marker, CA-125 marker and muellerian inhibitory substance (MIS) receptor type II, sTn (sialylated Tn antigen; TAG-72), FAP (fibroblast activation antigen), endosialin, EGFRVIII, LG, SAS, CD63, or any combination thereof.

9. The theranostic composition of claim 1, wherein:
(a) the bispecific ligand comprises Arg-Gly-Asp (RGD) peptide, bombesin (BBN)/gastrin-releasing peptide (GRP), cholecystokinin (CCK)/gastrin peptide, α-melanocyte-stimulating hormone (α-MSH), neuropeptide Y (NPY), neutrotensin (NT), [⁶⁸Ga]Ga-PSMA-HBED-CC ([⁶⁸Ga]Ga-PSMA-11 [PET]), [¹⁷⁷Lu]Lu/[⁹⁰Y]Y-J591, [¹²³I]I-MIP-1072, [¹³¹I]I-MIP-1095, ⁶⁸Ga or ¹⁷⁷Lu labeled PSMA-I&T, ⁶⁸Ga or ¹⁷⁷Lu labeled DKFZ-PSMA-617 (PSMA-617), somatostatin (SST) peptide, substance P, T140, tumor molecular targeted peptide 1 (TMTP1), vasoactive intestinal peptide (VIP), or any combination thereof, and/or
(b) the second arm of the bispecific ligand comprises the Arg-Gly-Asp (RGD) peptide, bombesin (BBN)/gastrin-releasing peptide (GRP), cholecystokinin (CCK)/gastrin peptide, α-melanocyte-stimulating hormone (α-MSH), neuropeptide Y (NPY), neutrotensin (NT), [⁶⁸Ga]Ga-PSMA-HBED-CC ([⁶⁸Ga]Ga-PSMA-11 [PET]), [¹⁷⁷Lu]Lu/[90Y]Y-J591, [¹²³I]-MIP-1072, [¹³¹I]I-MIP-1095, ⁶⁸Ga or ¹⁷⁷Lu labeled PSMA-I&T, ⁶⁸Ga or ¹⁷⁷Lu labeled DKFZ-PSMA-617 (PSMA-617), somatostatin (SST) peptide, substance P, T140, tumor molecular targeted peptide 1 (TMTP1), vasoactive intestinal peptide (VIP), or any combination thereof.

10. The theranostic composition of claim 1, wherein the first minicell surface component and/or the second minicell surface component comprises a lipopolysaccharide (LPS), and optionally wherein an O-polysaccharide of the lipopolysaccharide is radiolabeled.

11. The theranostic composition of claim 1, wherein the anti-neoplastic agent:
(a) comprises a super-cytotoxic drug; and/or
(b) comprises a super-cytotoxic drug, wherein the super-cytotoxic drug has a LD₅₀ that is lower than the ED₅₀ of the super-cytotoxic drug for a targeted cancer; and/or
(c) comprises a super-cytotoxic drug, wherein the minicell comprises from about 5×10⁵ to about 1.5×10⁶ molecules of the super-cytotoxic drug; and/or
(d) comprises a super-cytotoxic drug, wherein the supertoxic drug is PNU-159682; and/or
(e) comprises a compound selected from the group consisting of actinomycin-D, alkeran, ara-C, anastrozole, BiCNU, bicalutamide, bleomycin, busulfan, capecitabine, carboplatin, carboplatinum, carmustine, CCNU, chlorambucil, cisplatin, cladribine, CPT-11, cyclophosphamide, cytarabine, cytosine arabinoside, cytoxan, dacarbazine, dactinomycin, daunorubicin, dexrazoxane, docetaxel, doxorubicin, DTIC, epirubicin, ethyleneimine, etoposide, floxuridine, fludarabine, fluorouracil, flutamide, fotemustine, gemcitabine, hexamethylamine, hydroxyurea, idarubicin, ifosfamide, irinotecan, lomustine, mechlorethamine, melphalan, mercaptopurine, methotrexate, mitomycin, mitotane, mitoxantrone, oxaliplatin, paclitaxel, pamidronate, pentostatin, plicamycin, procarbazine, steroids, streptozocin, STI-571, tamoxifen, temozolomide, teniposide, tetrazine, thioguanine, thiotepa, tomudex, topotecan, treosulphan, trimetrexate, vinblastine, vincristine, vindesine, vinorelbine, VP-16, xeloda, asparaginase, AIN-457, bapineuzumab, belimumab, brentuximab, briakinumab, canakinumab, cetuximab, dalotuzumab, denosumab, epratuzumab, estafenatox, farletuzumab, figitumumab, galiximab, gemtuzumab, girentuximab (WX-G250), herceptin, ibritumomab, inotuzumab, ipilimumab, mepolizumab, muromonab-CD3, naptumomab, necitumumab, nimotuzumab, ocrelizumab, ofatumumab, otelixizumab, ozogamicin, pagibaximab, panitumumab, pertuzumab, ramucirumab, reslizumab, rituximab, REGN88, solanezumab, tanezumab, teplizumab, tiuxetan, tositumomab, trastuzumab, tremelimumab, vedolizumab, zalutumumab, zanolimumab, 5FC, accutane hoffmann-la roche, AEE788 novartis, AMG-102, anti neoplaston, AQ4N (Banoxantrone), AVANDIA (Rosiglitazone Maleate), avastin (Bevacizumab) genetech, BCNU, biCNU carmustine, CCI-779, CCNU, CCNU lomustine, celecoxib (Systemic), chloroquine, cilengitide (EMD 121974), CPT-11 (CAMPTOSAR, Irinotecan), dasatinib (BMS-354825, Sprycel), dendritic cell therapy, etoposide (Eposin, Etopophos, Vepesid), GDC-0449, gleevec (imatinib mesylate), gliadel wafer, hydroxychloroquine, IL-13, IMC-3G3, immune therapy, iressa (ZD-1839), lapatinib (GW572016), methotrexate for cancer (Systemic), novocure, OSI-774, PCV, RAD001 novartis (mTOR inhibitor), rapamycin (Rapamune, Sirolimus), RMP-7, RTA 744, simvastatin, sirolimus, sorafenib, SU-101, SU5416 sugen, sulfasalazine (Azulfidine), sutent (Pfizer), TARCEVA (erlotinib HCl), taxol, TEMODAR schering-plough, TGF-B anti-sense, thalomid (thalidomide), topotecan (Systemic), VEGF trap, VEGF-trap, vorinostat (SAHA), XL 765, XL184, XL765, zarnestra (tipifarnib), ZOCOR (simvastatin), cyclophosphamide (Cytoxan), (Alkeran), chlorambucil (Leukeran), thiopeta (Thioplex), busulfan (Myleran), procarbazine (Matulane), dacarbazine (DTIC), altretamine (Hexalen), clorambucil, cisplatin (Platinol), ifosafamide, methotrexate (MTX), 6-thiopurines (Mercaptopurine [6-MP], Thioguanine [6-TG]), mercaptopurine (Purinethol), fludarabine phosphate, (Leustatin), flurouracil (5-FU), cytarabine (ara-C), azacitidine, vinblastine (Velban), vincristine (Oncovin), podophyllotoxins (etoposide {VP-16} and teniposide {VM-26}), camptothecins (topotecan and irinotecan), taxanes such as paclitaxel (Taxol) and docetaxel (Taxotere), (Adriamycin, Rubex, Doxil), dactinomycin (Cosmegen), plicamycin (Mithramycin), mitomycin: (Mutamycin), bleomycin (Blenoxane), estrogen and androgen inhibitors (Tamoxifen), gonadotropin-releasing hormone agonists (Leuprolide and Goserelin (Zoladex)), aromatase inhibitors (Aminoglutethimide and Anastrozole (Arimidex)), amsacrine, asparaginase (El-spar), mitoxantrone (Novantrone), mitotane (Lysodren), retinoic acid derivatives, bone marrow growth factors (sargramostim and filgrastim), amifostine, pemetrexed, decitabine, iniparib, olaparib, veliparib, everolimus, vorinostat, entinostat (SNDX-275), mocetinostat (MGCD0103), panobinostat (LBH589), romidepsin, valproic acid, flavopiridol, olomoucine, roscovitine, kenpaullone, AG-024322 (Pfizer), fascaplysin, ryuvidine, purvalanol A, NU2058, BML-259, SU 9516, PD-0332991, P276-00, geldanamycin, tanespimycin, alvespimycin, radicicol, deguelin, BIIB021, cis-imidazoline, benzodiazepinedione, spiro-oxindoles, isoquinolinone, thiophene, 5-deazaflavin, tryptamine, aminopyridine, diaminopyrimidine, pyridoisoquinoline, pyrrolopyrazole, indolocarbazole, pyrrolopyrimidine, dianilinopyrimidine, benzamide, phthalazinone, tricyclic indole, benzimidazole, indazole, pyrrolocarbazole, isoindolinone, morpholinyl anthracycline, a maytansinoid, ducarmycin, auristatins, calicheamicins (DNA damaging agents), α-amanitin (RNA polymerase II inhibitor), centanamycin, pyrrolobenzodiazepine, streptonigtin, nitrogen mustards, nitrosorueas, alkane sulfonates, pyrimidine analogs, purine analogs, antimetabolites, folate analogs, anthracyclines, taxanes, vinca alkaloids, topoisomerase inhibitors, hormonal agents, and any combination thereof, and/or
(f) comprises a functional nucleic acid or a polynucleotide encoding a functional nucleic acid; and/or
(g) comprises a functional nucleic acid, wherein the functional nucleic acid inhibits a gene that promotes tumor cell proliferation, angiogenesis or resistance to chemotherapy and/or that inhibits apoptosis or cell cycle arrest; and/or
(h) comprises a functional nucleic acid, wherein the functional nucleic acid is selected from the group consisting of siRNA, miRNA, shRNA, lincRNA, antisense RNA, and ribozyme; and/or
(i) comprises a polynucleotide encoding a gene that promotes apoptosis.

12. The theranostic composition of claim 1, wherein:
(a) the first surface component comprises a first polypeptide and the second surface component comprises a second polypeptide, wherein the first polypeptide and the second polypeptide share greater than 90% sequence identity with each other; and/or
(b) the first surface component comprises a first polypeptide and the second surface component comprises a second polypeptide, wherein the first polypeptide and the second polypeptide have less than 90% sequence identity with each other.

13. A method:
(a) of imaging a tumor in a subject comprising administering systemically to the subject the theranostic composition of claim 1, wherein the composition comprises a diagnostically effective amount of the radio-imaging agent; or
(b) for treating a tumor in a subject in need, comprising administering systemically to the subject the theranostic composition of claim 1, wherein the composition comprises a therapeutically effective amount of the radio-imaging agent and a therapeutically effective amount of the anti-neoplastic agent; or
(c) of imaging and treating a tumor in a subject in need, comprising administering systemically to the subject the theranostic composition of claim 1, wherein the composition comprises:
(i) a diagnostically effective amount of the radio-imaging agent, wherein the amount of the radio-imaging agent is also therapeutically effective; and
(ii) a therapeutically effective amount of the anti-neoplastic agent; or
(d) of adjusting the signal intensity of an imaged tumor in a subject comprising:
(i) systemically administering a first dose of a theranostic composition according to claim 1 followed by imaging the tumor;
(ii) systemically administering a second dose of a theranostic composition according to claim 1 followed by imaging the tumor, wherein:
(A) the second dose of a theranostic composition comprises a greater amount of the radio-imaging agent per minicell as compared to the first dose; or
(B) the second dose of a theranostic composition comprises a lesser amount of the radio-imaging agent per minicell as compared to the first dose; and then
(iii) comparing the imaging results following (a) and (b) to obtain the adjusted signal intensity.

14. The method of claim 13, wherein the tumor does not comprise:
(a) a brain tumor; and/or
(b) the tumor does not comprise a glioblastoma, astrocytic tumor, oligodendroglial tumor, ependymoma, craniopharyngioma, pituitary tumor, primary lymphoma of the brain, pineal gland tumor, primary germ cell tumor of the brain, or combination thereof; and/or
(b) the tumor does not comprise a spleen tumor or a liver tumor.

15. The method of claim 13, wherein:
(a) the plurality of the purified, intact bacterially derived minicells comprises at least about 10⁸ minicells; and/or
(b) the subject is a human.

16. The method of claim 14, wherein:
(a) the plurality of the purified, intact bacterially derived minicells comprises at least about 10⁸ minicells; and/or
(b) the subject is a human.
